SEQUENCE LISTING (2 sequences)

SEQ ID NO: 1 - DNA, 21 bp, Artificial Sequence
  Description of Artificial Sequencehuman keratin 14 (K14) small interfering RNA (siRNA) sense strand
gaguugaaccugcgcaugatt

SEQ ID NO: 2 - DNA, 21 bp, Artificial Sequence
  Description of Artificial Sequencehuman keratin 14 (K14) small interfering RNA (siRNA) antisense strand
ucaugcgcagguucaacuctg

(19) United States
(12) Patent Application Publication
(10) Pub. No.: US 2012/0190573 A1
(43) Pub. Date: Jul. 26, 2012

(54) BIOMARKER OF LUNG CANCER

(75) Inventors: Brigitte N. Gomperts, Los Angeles, CA (US); Aik T. Ooi, Los Angeles, CA (US); Derek W. Nickerson, Santa Monica, CA (US); Lee Goodglick, Los Angeles, CA (US)

(73) Assignee: The Regents of the University of California, Oakland, CA (US)

(21) Appl. No.: 13/378,654

(22) PCT Filed: Jul. 27, 2010
(86) PCT No.: PCT/US10/43438

§ 371 Date: Feb. 27, 2012

Related U.S. Application Data
(60) Provisional application No. 61/228,933, filed on Jul. 27, 2009.

(51) Int. Cl. C40B 30/04; C12Q 1/68; G01N 27/62; G01N 33/566; G01N 21/64

(52) U.S. Cl. 506/9; 435/7.1; 435/7.92; 435/29; 435/6.12

(57) ABSTRACT

The present invention provides methods of providing a prognosis for a lung cancer in a subject and methods of predicting the risk of metastasis of a lung cancer in a subject. The present invention additionally provides kits that find use in the practice of the methods of the invention.

### CROSS-REFERENCES TO RELATED APPLICATIONS

This application claims priority to U.S. Provisional Application Ser. No. 61/228,933, filed on Jul. 27, 2009, which is incorporated herein by reference in its entirety.

### STATEMENT AS TO RIGHTS TO INVENTIONS MADE UNDER FEDERALLY SPONSORED RESEARCH OR DEVELOPMENT

This invention was made with Government support of Grant Nos. CA086366 and HL074229, awarded by the National Institutes of Health. The Government has certain rights in this invention.

### REFERENCE TO A “SEQUENCE LISTING,” SUBMITTED ELECTRONICALLY

The sequence listing contained in the file named “008074-5030 Sequence Listing.txt”, created on Aug. 17, 2010 and having a size of 4.0 kilobytes, has been submitted electronically herewith via EFS-Web, and the contents of the txt file are hereby incorporated by reference in their entirety.

### BACKGROUND OF THE INVENTION

Lung cancer is a disease that accounts for an estimated 222,000 cases and 157,000 deaths in the United States each year. Lung cancer is the leading cause of cancer death for both men and women, accounting for about 28% of all cancer deaths. The disease is classified into two major categories, non-small cell lung cancer (NSCLC), which accounts for about 85% of lung cancer cases, and small cell lung cancer (SCLC). In NSCLC, the most common forms are adenocarcinoma, squamous cell carcinoma, and large cell carcinoma, although other types occur less frequently.

There are four stages of NSCLC, stages I through IV, that are determined based on tumor size, the amount of tumor that has spread to nearby lymph nodes, and the presence or absence of distant metastases. The overall prognosis for NSCLC varies by stage; while the five-year survival rate for stage I disease is about 60-70%, patients with a diagnosis of metastatic non-small cell lung cancer have a median survival of just 4-5 months [1].

Lung cancer is a heterogeneous disease and the natural history is still not well understood. The classic exponential growth model of tumor metastasis may not be relevant in some tumors, where the biology of the disease may impact prognosis more than the time and size of growth of the tumor [2]. For example, as many as 40% of patients with completely resected stage I NSCLC will experience a recurrence of their disease, which suggests that a subpopulation of cells in these tumors is more prone to micrometastatic behavior [2].

The cancer stem cell (CSC) model of tumor development and progression refers to the presence of a population of rare cells in a tumor that have stem cell properties, namely they are capable of self-renewal and differentiation to their progeny. In this model, the self-renewal capacity of the CSCs is responsible for maintaining tumor growth indefinitely and the other cells that make up most of the tumor are actively proliferating and differentiating and therefore susceptible to current conventional cancer therapies [3-10]. Consistent with this model, CSCs would be considered to be tumor-initiating cells [3-10]. Recently, it has been found that CSCs may not necessarily represent rare cells in a tumor and that the tumor-initiating cell in a cancer reflects a cell with the property of indefinite self-renewal, which could be a rare stem cell, a progenitor cell, or a differentiated cell that has developed the ability to self-renew [11]. These tumor-initiating cells are thought to arise from cells that have dysregulated repair resulting in indefinite self-renewal and are associated with relapse and recurrence of cancers and a poor prognosis, presumably due to resistance to chemotherapy and radiotherapy [3, 5-10]. This model of CSCs leading to tumor resistance fits well with the natural history of lung cancer, with its high incidence of recurrence and metastasis.

Although there is a limited understanding of stem and progenitor cells in the proximal airway epithelium, some populations have been identified with self-renewing and differentiation properties [12-15]. Keratin 5 (K5)-expressing basal cells are considered to be progenitor cells in the adult large airways at steady state and during airway epithelial repair [12-15]. In humans, unlike mice, K5-expressing basal cells have been found throughout the tracheobronchial tree [12]. It was previously thought that K14 is the obligate intermediate filament-binding partner of K5 in the basal cells of the airway epithelium [12, 16]. However, although K14+ progenitor epithelial cells in the airway are important for repair, K14+ cells are rarely found in the airway epithelium under homeostatic conditions while K5+ cells are relatively abundant [12, 16]. The absence of K14 expression in airway epithelium following the completion of normal repair suggests that K14 expression is tightly regulated at steady state.

Because it is believed that cells having dysregulated repair give rise to tumor-initiating cells and are associated with relapse and recurrence of cancers, the identification of markers that are associated with cells having dysregulated repair will likely be useful for developing methods of diagnosing cancers and predicting the prognosis of cancers. The present invention addresses this need and others.

### BRIEF SUMMARY OF THE INVENTION

In one aspect, the present invention provides a method of providing a prognosis for a lung cancer in a subject, the method comprising the steps of: (a) analyzing a sample from the subject with an assay that specifically detects keratin 14 (K14)-expressing cells; and (b) determining whether or not the number of K14-expressing cells in the sample is increased as compared to a control; thereby providing the prognosis for the lung cancer.

In one embodiment, an increased number of K14-expressing cells in the sample as compared to the control indicates a poor prognosis.

In one embodiment, the method further comprises the step of determining whether the number of K14-expressing cells in the sample is at least 5% of the total number of cells in the sample, wherein determining that at least 5% of the total number of cells in the sample are K14-expressing cells indicates a poor prognosis for the lung cancer.

In another aspect, the present invention provides a method of providing a prognosis for a lung cancer in a subject, the method comprising the steps of: (a) analyzing a sample from the subject with an assay that specifically detects keratin 14 (K14) expression; and (b) determining whether the level of K14 expression in the sample is increased as compared to the level of K14 expression in a control; thereby providing the prognosis for the lung cancer.

In one embodiment, an increased number of K14-expressing cells in the sample as compared to the control indicates a poor prognosis.

In another aspect, the present invention provides a method of predicting the risk of metastasis of a lung cancer in a subject, the method comprising the steps of: (a) analyzing a sample from the subject with an assay that specifically detects keratin 14 (K14)-expressing cells; and (b) determining whether or not the number of K14-expressing cells in the sample is increased as compared to a control; thereby predicting the risk of metastasis of the lung cancer.

In one embodiment, an increased number of K14-expressing cells in the sample as compared to the control indicates a higher risk of metastasis of the lung cancer.

In one embodiment, the lung cancer is a non-small cell lung cancer. In one embodiment, the lung cancer is a squamous cell carcinoma.

In one embodiment, the assay detects protein and is ELISA, Western blotting, flow cytometry, immunofluorescene, immunohistochemistry, or mass spectroscopy. In one embodiment, the assay comprises a reagent that binds to a protein. In one embodiment, the reagent is an antibody. In one embodiment, the reagent is a monoclonal antibody.

In one embodiment, the assay detects nucleic acid and is mass spectroscopy, PCR, microarray hybridization, thermal cycle sequencing, capillary array sequencing, or solid phase sequencing. In one embodiment, the reagent is a nucleic acid. In one embodiment, the reagent is an oligonucleotide. In one embodiment, the reagent is a RT-PCR primer set.

In one embodiment, the assay detects a K14-expressing cell that also expresses keratin 5 (K5).

In one embodiment, the sample is from lung tissue, a lung tumor biopsy, a lymph node biopsy, an adrenal tumor biopsy, a liver tumor biopsy, a brain tumor biopsy, or a bone tumor biopsy.

In one embodiment, the subject has a history of smoking.

In yet another aspect, the present invention provides a kit for use in providing a prognosis for a lung cancer in a subject, the kit comprising a reagent that specifically binds to a keratin 14 (K14)-expressing cell.

In one embodiment, the reagent is an antibody. In one embodiment, the antibody is labeled. In one embodiment, the kit further comprises additional reagents for detection of the labeled antibody.

In one embodiment, the reagent is a nucleic acid. In one embodiment, the reagent is an RT-PCR primer.

### BRIEF DESCRIPTION OF THE DRAWINGS

FIG. 1. Keratin 14 (K14) and keratin 5 (K5)-expressing progenitor cell populations in the airway epithelium at steady state and during repair. A, B. Representative sections of immunofluorescent staining identifies cells in the submucosal glands and submucosal gland duct that express K14 (Alexa fluor 488, green) and K5 (Cy3, red). Basal cells of the pseudostratified columnar airway epithelium express K5 but do not express K14. A) is representative of immunostaining seen in mice (scale bar=20 μm), B) is representative of staining in humans (scale bar=100 μm). H&E stained representative sections are included to demonstrate the anatomy of the pseudostratified columnar airway epithelium (arrow), the submucosal glands (dotted arrow) and submucosal gland ducts (dashed arrow).

FIG. 2. Immunofluorescence staining for K14 and K5. A. Representative sections of immunofluorescent staining of K14 (Alexa fluor 488, green) and K5 (Cy3, red) expressing cells in the mouse tracheal airway epithelium after hypoxic-ischemic injury from tracheal transplantation. i. K14 and K5-expressing cells are seen in the submucosal glands, submucosal gland ducts and repairing surface airway epithelium. ii. K14 and K5-expressing cells are seen on the repairing surface airway epithelium. iii. K14 and K5-expressing cells are seen in a hyperplastic area of repairing surface airway epithelium but in areas of pseudostratified columnar epithelium K5 expression is present in the basal cells but K14 expression is absent. iv. Repaired pseudostratified columnar epithelium with K5 expression in the basal cells and absence of K14 expression. Corresponding H&E sections are included to demonstrate the histopathology of the repairing airway. B. Representative sections of immunofluorescent staining of K14 (Alexa fluor 488, green) and K5 (Cy3, red) expressing cells in repairing airway epithelial human tissue from smokers with reserve cell hyperplasia and squamous metaplasia. K5+K14− basal cells are seen in normal airway epithelium (red arrow). A few K14+K5+ few basal cells are also present (yellow arrow). K14+K5+ cells are seen in an area of reserve cell hyperplasia and in squamous metaplasia (green arrows). H&E staining of the section demonstrates the areas of normal pseudostratified columnar epithelium (arrows), reserve cell hyperplasia (dotted arrow), and squamous metaplasia (dashed arrow), (scale bar=20 μm). C. Representative sections of immunofluorescent staining of K14 (Alexa fluor 488, green) and K5 (Cy3, red) expressing cells in repairing airway epithelial human tissue from smokers with dysplasia and carcinoma in situ lesions. K14+K5+ cells are seen in areas of moderate dysplasia (green arrows) and carcinoma in situ (severe dysplasia)(green dashed arrow). H&E staining of the section demonstrates the areas of moderate dysplasia (arrows) and carcinoma in situ (severe dysplasia) (dashed arrow), (scale bar=20 μm).

FIG. 3. Spot expression levels for K5 and K14 positivity in the lung cancer tissue microarray by NSCLC histologic subtype. A. In the majority of squamous carcinomas, nearly all the tumor cells show K5 staining, in sharp contrast to adenocarcinomas and large cell carcinoma. Adenosquamous carcinomas are somewhat intermediate. B. Most tumors are negative for K14 staining. Median percentage positivity was zero in tumors other than squamous carcinoma.

FIG. 4. Analysis of K14 mRNA and protein expression in adenocarcinoma and squamous lung cancer samples. A. Quantitative real-time PCR For K14 mRNA expression was performed on RNA extracted from frozen tissue sections of archived human adenocardinomas and squamous lung cancers. K14 expression was detected in all samples examined K14 expression in sample #10 is normalized to 1. K14 expression in squamous lung cancer correlated with K14 expression seen by immunostaining in two squamous lung cancer samples—sample #0 with low K14 expression and sample #11 with high K14 expression. Samples 1-9 are all adenocarcinomas. B. Western blot analysis of squamous lung cancer (SCC) and lung adenocarcinoma (AC) patient samples revealed the presence of K14 protein in all NSCLC tumors examined C. Analysis of two published mRNA expression cohorts of NSCLC patients revealed distributions of K14 expression in different histologic subtypes of NSCLC. K14 mRNA expression was greater in squamous lung cancer (SLC), but still present in other histologic subtypes at varying expression levels, similar to that seen in the tissue microarray protein expression data (FIG. 3).

FIG. 5. Kaplan Meier Survival curves showing that K14 expression in NSCLC correlates with poor prognosis. A. Analysis of the UCLA TMA revealed that patients with NSCLC that expressed K14 had a significantly worse prognosis than patients with NSCLC in which K14 was below the level of detection (P=0.004, hazard ratio=1.58). B. Analysis of the M.D. Anderson TMA also showed that patients with NSCLC that expressed K14 had a worse prognosis than patients with NSCLC in which K14 was below the level of detection (P=0.003, hazard ration=1.60).

FIG. 6. Kaplan Meier Survival curves from the UCLA TMA showing that the poor prognosis related to K14-expressing NSCLC tumors correlated with smoking. A. In all smokers (current and former) K14 positivity in NSCLC tumors had the highest predictive value of death from NSCLC (P=0.0009, hazard ratio=1.77, n=332). B. The predictive value of K14 expressing NSCLC tumors in individuals who were current smokers (P=0.01, hazard ratio=2.11, n=124). C. K14 positivity was still somewhat predictive of death due to disease in former smokers as well (P=0.04, hazard ratio=1.68, n=157). D. In never smokers, the presence of K14+ cells had no predictive value for outcome (P=0.93, hazard ratio=0.95, n=53).

FIG. 7. Kaplan Meier Survival curves from the M.D. Anderson Cancer Center TMA showing that the poor prognosis related to K14 expressing NSCLC tumors also correlates with smoking. A. In all smokers (current and former) K14 positivity in NSCLC tumors predicted a worse prognosis (P=0.004). B. In never smokers, the presence of K14+ cells had no predictive value for outcome (P=0.356).

FIG. 8. Analysis of K5 and K14 expressing NSCLCs from the UCLA TMA. NSCLC tumors that expressed both K5 and K14 compared to those that were K5 and/or K14 negative revealed a significantly worse prognosis in NSCLC patients with K5+K14+ tumors (P=0.002), and again this was especially significant in smokers (P=0.0007).

FIG. 9. Mean percentage of K14+ cells in distant metastases relative to primary tumor and lymph node sites. There was a significant increase in the percentage of K14+ cells in metastases compared to the primary sites in squamous lung cancer (SLC) (P<0.001), but not in other histologic subtypes.

FIG. 10. Dual immunofluorescent staining of human premalignant lesions and tumors to assess populations of proliferating cells that also express K14. i.-ii. Dual immunofluorescent staining of premalignant lesions for K14 and PCNA. In premalignant lesions we found that 57.8%±5.1% of K14+ cells also expressed PCNA. iii.-iv. Dual immunofluorescent staining of tissue from SCC for K14 and PCNA. In SCC we found that 67.3%±7.3% of K14+ cells also expressed PCNA.

FIG. 11. K14 knockdown studies in BEAS2B immortalized normal human bronchial epithelial cells to assess the effect on proliferation. A. Western blot analysis demonstrating a 90% reduction in K14 expression in BEAS2B cells 5 days after transfection with an siRNA for K14 versus a control siRNA. PCNA expression in transfected cells was found to be equivalent by Western blot analysis in K14 siRNA and control siRNA transfected cells. B. The MTS proliferation assay showed no effect on cell proliferation in the K14 siRNA transfected cells compared to the control siRNA transfected cells.

FIG. 12. Transient overexpression of K14 in an immortalized normal human bronchial epithelial cell line (BEAS-2B) resulted in increased motility of the cells in a wound healing assay.

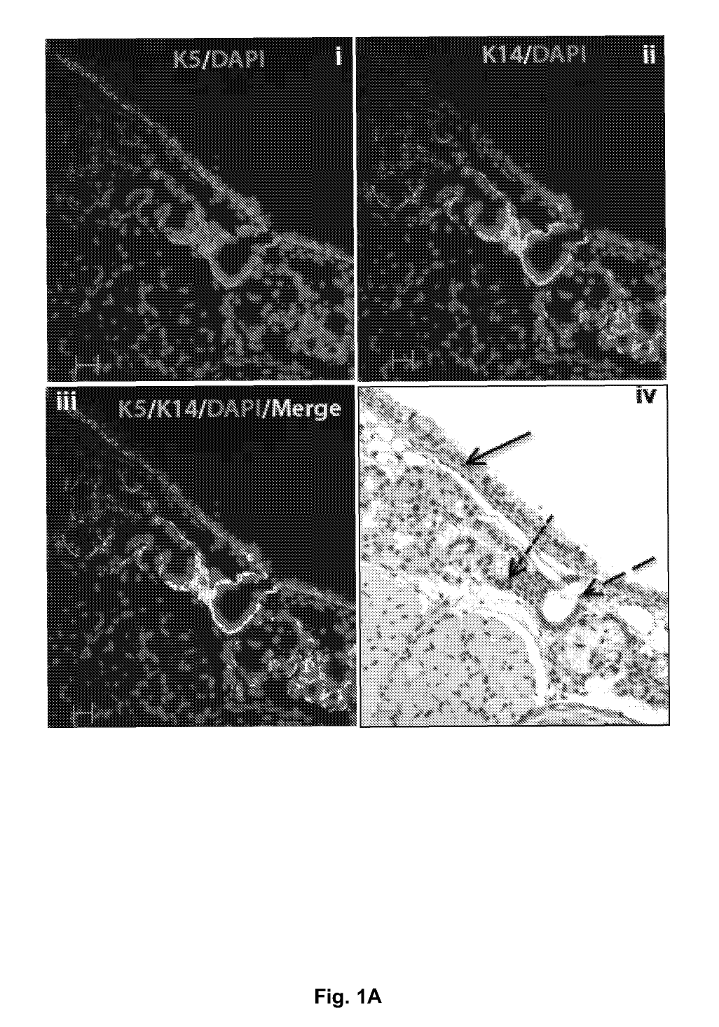

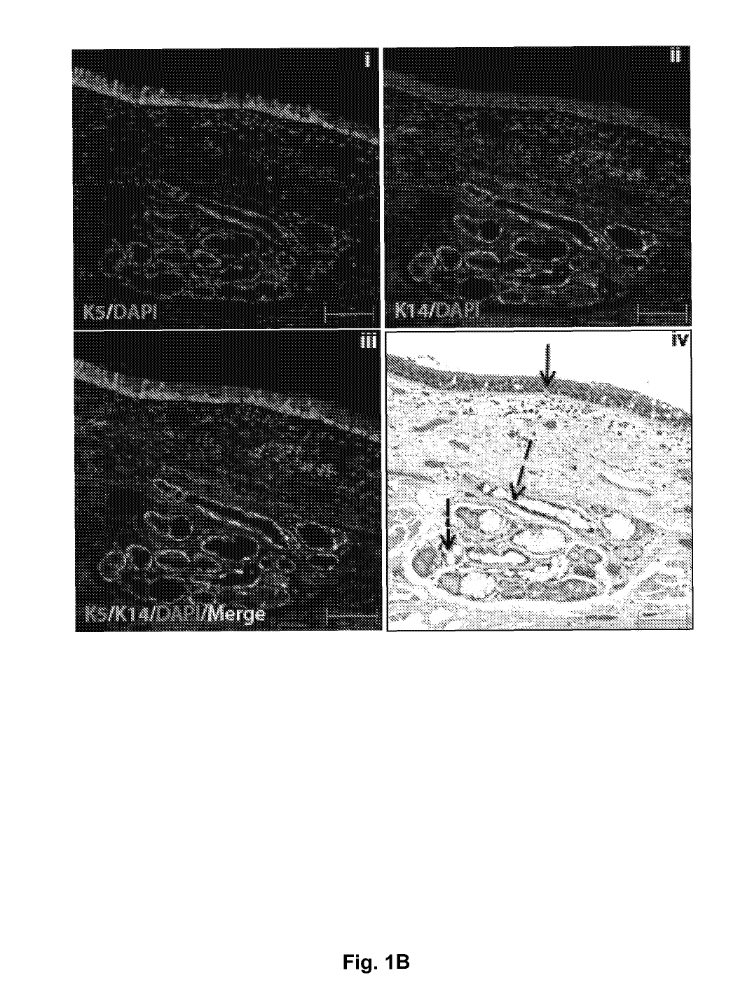

### DETAILED DESCRIPTION OF THE INVENTION

### I. Introduction

The present invention relates to the discovery that persistence of keratin 14 (K14) expression is found in aberrant repair with premalignant lesions and in a subset of NSCLCs associated with injury from smoking. We have found that the presence of dysregulated K14+ progenitor cells in NSCLC after chronic smoking injury is associated with increased mortality from lung cancer. Additionally, we have found that the presence of K14+ cells in the primary tumors of smokers is associated with metastatic disease.

Accordingly, the present invention provides methods of providing a prognosis for lung cancer in a subject by determining whether the number of K14-expressing cells in a sample from the subject is increased as compared to a control or by determining whether the level of K14 expression in a sample from the subject is increased as compared to the level of K14 expression in a control. The present invention also provides methods of predicting the risk of metastasis of a lung cancer in a subject by determining whether the number of K14-expressing cells in a sample from the subject is increased as compared to a control.

### II. Definitions

As used herein, the following terms have the meanings ascribed to them unless specified otherwise.

The term “cancer” refers to human cancers and carcinomas, sarcomas, adenocarcinomas, lymphomas, leukemias, solid and lymphoid cancers, etc. Examples of different types of cancer include, but are not limited to, lung cancer, pancreatic cancer, breast cancer, gastric cancer, bladder cancer, oral cancer, ovarian cancer, thyroid cancer, prostate cancer, uterine cancer, testicular cancer, neuroblastoma, squamous cell carcinoma of the head, neck, cervix and vagina, multiple myeloma, soft tissue and osteogenic sarcoma, colorectal cancer, liver cancer (i.e., hepatocarcinoma), renal cancer (i.e., renal cell carcinoma), pleural cancer, cervical cancer, anal cancer, bile duct cancer, gastrointestinal carcinoid tumors, esophageal cancer, gall bladder cancer, small intestine cancer, cancer of the central nervous system, skin cancer, choriocarcinoma; osteogenic sarcoma, fibrosarcoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, and acute myelocytic leukemia. Cancers embraced in the current application include both metastatic and non-metastatic cancers.

As used herein, the term “lung cancer” refers to a group of malignant or neoplastic cancers originating in the lung of an individual. Non-limiting examples of lung cancer include non-small cell lung cancer (NSCLC) (e.g., squamous cell carcinoma, adenocarcinoma, and large cell carcinoma), small cell lung cancer (SCLC) or “oat cell” carcinoma, combined small cell carcinoma, carcinoid, adenosquamous carcinoma, sarcomatoid carcinoma, and adenoid cystic carcinoma.

As used herein, “providing a prognosis” refers to providing a prediction of the likelihood of metastasis, predictions of disease free and overall survival, the probable course and outcome of cancer therapy, or the likelihood of recovery from the cancer, in a subject. A “poor prognosis,” as used herein, refers to an increased risk of long-term mortality.

As used herein, “metastasis” refers to spread of a cancer from the primary tumor or origin to other tissues and parts of the body, such as lymph node, lung, adrenal gland, liver, brain, and/or bone.

The “level of expression” of a marker refers to the amount of protein or nucleic acid (RNA) that is transcribed or translated in a cell. The level of expression of a marker in a sample (e.g., a cancer cell or tumor sample from a subject having lung cancer) can be detected and quantitated in comparison to a “control,” a non-cancerous cell or tissue, and the level of expression of a marker in a sample is “increased” relative to a control if the protein or nucleic acid is transcribed or translated at a detectably greater level in the sample as compared to the control. Increased expression includes increases in expression due to transcription, post transcriptional processing, translation, post-translational processing, cellular localization (e.g., organelle, cytoplasm, nucleus, cell surface), and RNA and protein stability, as compared to a control (e.g., non-cancerous) cell. Expression of a marker can be detected using conventional techniques for detecting mRNA (i.e., RT-PCR, PCR, hybridization) or proteins (i.e., ELISA, immunohistochemical techniques). Increased expression can be 10%, 20%, 30%, 40%, 50%, 60%, 70%, 80%, 90% or more in comparison to a control cell. In certain instances, increased expression is 1-fold, 2-fold, 3-fold, 4-fold or more higher levels of transcription or translation in comparison to a control cell.

Expression of a marker can be detected for a tissue (e.g., lung epithelium), lesion, or tumor and quantitated as the number of cells in the tissue, lesion, or tumor that express the marker at a detectable level in comparison to a control (non-cancerous) tissue. As used herein, the number of marker-expressing cells in a sample is “increased” relative to a control if the number of cells expressing the marker at a detectable level in the sample is greater than the number of cells expressing the marker at a detectable level in the control. Increased number of marker-expressing cells can be 10%, 20%, 30%, 40%, 50%, 60%, 70%, 80%, 90% or more in comparison to a control tissue. Alternatively, the number of marker-expressing cells in a tissue, lesion, or tumor can be quantitated as a percentage of the total number of cells in the tissue, lesion, or tumor. In some embodiments, a tissue, lesion, or tumor is said to express the marker or be positive for the marker if a threshold percentage of cells express the marker at a detectable level. In some embodiment, a tissue, lesion, or tumor is said to express the marker or be positive for the marker if at least 5% of the cells in the tissue, lesion, or tumor express the marker at a detectable level.

Markers of the present invention include keratin 14 (K14) and keratin 5 (K5). The terms “keratin 14” and “keratin 5” refer to nucleic acids and polypeptide polymorphic variants, alleles, mutants, and interspecies homologs that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a region of over a region of at least about 25, 50, 100, 200, 500, 1000, or more amino acids, to an amino acid sequence encoded by a keratin 14 or keratin 5 nucleic acid (Accession numbers NM_{—}000526 and NM_{—}000424) or amino acid sequence of a keratin 14 or keratin 5 protein (NP_{—}000517 and NP_{—}000415); (2) bind to antibodies, e.g., polyclonal antibodies, raised against an immunogen comprising an amino acid sequence of a keratin 14 or keratin 5 protein and conservatively modified variants thereof; (3) specifically hybridize under stringent hybridization conditions to an anti-sense strand corresponding to a nucleic acid sequence encoding a keratin 14 or keratin 5 protein and conservatively modified variants thereof; (4) have a nucleic acid sequence that has greater than about 95%, preferably greater than about 96%, 97%, 98%, 99%, or higher nucleotide sequence identity, preferably over a region of at least about 25, 50, 100, 200, 500, 1000, or more nucleotides, to a keratin 14 or keratin 5 nucleic acid. A polynucleotide or polypeptide sequence is typically from a mammal including, but not limited to, primate, e.g., human; rodent, e.g., rat, mouse, hamster; cow, pig, horse, sheep, or any mammal. The nucleic acids and proteins of the invention include both naturally occurring or recombinant molecules.

The terms “identical” or “percent identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math., 2:482 (1981), by the homology alignment algorithm of Needleman & Wunsch, J. Mol. Biol., 48:443 (1970), by the search for similarity method of Pearson & Lipman, Proc. Nat'l. Acad. Sci. USA, 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)).

A preferred example of algorithm that is suitable for determining percent sequence identity and sequence similarity are the BLAST and BLAST 2.0 algorithms, which are described in Altschul et al., Nuc. Acids Res., 25:3389-3402 (1977) and Altschul et al., J. Mol. Biol., 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA, 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). The term nucleic acid is used interchangeably with gene, cDNA, mRNA, oligonucleotide, and polynucleotide.

A particular nucleic acid sequence also implicitly encompasses “splice variants” and nucleic acid sequences encoding truncated forms of proteins. Similarly, a particular protein encoded by a nucleic acid implicitly encompasses any protein encoded by a splice variant or truncated form of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. Nucleic acids can be truncated at the 5′ end or at the 3′ end. Polypeptides can be truncated at the N-terminal end or the C-terminal end. Truncated versions of nucleic acid or polypeptide sequences can be naturally occurring or recombinantly created.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, ÿ-carboxyglutamate, and O-phosphoserine. Amino acid analogs refers to compounds that have the same basic chemical structure as a naturally occurring amino acid, i.e., any carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant,” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The phrase “specifically (or selectively) binds” or “specifically (or selectively) detects” refers to a binding reaction that is determinative of the presence of a marker, such as a protein or nucleic acid, which is often in a heterogeneous population of proteins or nucleic acids and other biologics. For example, the presence of a protein is specifically detected if, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Luminex® xMAP technology is particularly well suited for the present invention. Similarly, the presence of a nucleic acid is specifically detected if, under designated hybridization conditions, the specified oligonucleotides bind to a particular nucleic acid target sequence at least two times the background and more typically more than 10 to 100 times background. Specific binding to an oligonucleotide under such conditions requires an oligonucleotide that is selected for its specificity for a particular nucleic acid sequence. For example, oligonucleotides can be selected which bind to the target nucleic acid sequence under stringent hybridization conditions.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., Current Protocols in Molecular Biology, ed. Ausubel, et al., supra.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al., PCR Protocols, A Guide to Methods and Applications (Academic Press, Inc., N.Y., 1990).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. Antibodies can be polyclonal or monoclonal, derived from serum, a hybridoma or recombinantly cloned, and can also be chimeric, primatized, or humanized.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)₂′ a dimer of Fab which itself is a light chain joined to V_{H}-C_{H}1 by a disulfide bond. The F(ab)₂′ may be reduced under mild conditions to break the disulfide linkage in the hinge region, thereby converting the F(ab)₂′ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature, 348:552-554 (1990)).

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

“Biological sample” includes sections of tissues such as biopsy and autopsy samples, and frozen sections taken for histologic purposes. Such samples include blood and blood fractions or products (e.g., serum, plasma, platelets, red blood cells, and the like), sputum or saliva, tissue (e.g., lung tissue), cultured cells, e.g., primary cultures, explants, and transformed cells, tumors, stool, urine, etc. A biological sample is typically obtained from a “subject” such as a eukaryotic organism, most preferably a mammal such as a primate e.g., chimpanzee or human; cow; dog; cat; a rodent, e.g., guinea pig, rat, or mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., lung, colon, prostate, kidney, bladder, lymph node, liver, bone marrow, blood cell, etc.), the size and type of the tumor (e.g., solid or suspended, blood or ascites), among other factors. Representative biopsy techniques include, but are not limited to, excisional biopsy, incisional biopsy, needle biopsy, surgical biopsy, and bone marrow biopsy. An “excisional biopsy” refers to the removal of an entire tumor mass with a small margin of normal tissue surrounding it. An “incisional biopsy” refers to the removal of a wedge of tissue that includes a cross-sectional diameter of the tumor. A diagnosis or prognosis made by endoscopy or fluoroscopy can require a “core-needle biopsy” of the tumor mass, or a “fine-needle aspiration biopsy” which generally obtains a suspension of cells from within the tumor mass. Biopsy techniques are discussed, for example, in Harrison's Principles of Internal Medicine, Kasper, et al., eds., 16th ed., 2005, Chapter 70, and throughout Part V.

As used herein, the phrase “history of smoking” refers to an individual who has smoked at least 100 cigarettes over the course of his or her lifetime. The term “smoker” includes both current smokers (individuals who are smokers when the sample is collected or quit within one year of when the sample is collected) and former smokers (individuals who quit smoking more than one year from when the sample is collected).

### III. Prognostic Methods

The present invention provides methods of providing a prognosis for lung cancer in a subject by detecting the expression of keratin 14 (K14), which is expressed in increased levels and in a higher number of cells in lung cancer as compared to a normal (non-cancerous) tissue. The present invention also provides methods of predicting the risk of metastasis of lung cancer by detecting the expression of keratin 14. The methods can also be used to devise a suitable therapy for cancer treatment, e.g., by indicating whether or not the cancer is still at an early stage or if the cancer had advanced to a stage where aggressive therapy would be ineffective.

Prediction and prognosis involve determining the level of K14 polynucleotide or the corresponding polypeptide in a sample from a patient, or determining the number of K14-expressing cells in a sample from a patient, and then comparing the level of K14 expression or number of K14-expressing cells to a baseline or range. Typically, the baseline value is representative of levels of the polynucleotide or corresponding polypeptide, or number of cells expressing the polynucleotide or corresponding polypeptide, in a healthy person not suffering from, or destined to develop, lung cancer, as measured using a biological sample such as a lung biopsy or other tissue sample or bodily fluid sample (e.g., serum, blood, or saliva). Variation of levels of a marker of the present invention, or variation of number of marker-expressing cells, from the baseline range (either up or down) indicates that the patient has an increased or decreased risk of long term mortality.

In one embodiment, real-time or quantitative PCR is used to examine expression of K14 using RNA from a biological sample such as tumor tissue. RNA extraction can be performed by any method know to those of skill in the art, e.g., using Trizol® and RNeasy®. Real-time PCR can be performed by any method known to those of skill in the art, e.g., TaqMan® Real-Time PCR using Applied Biosystem assays. Gene expression is calculated relative to non-cancerous RNA, e.g., non-cancerous lung RNA, and expression is normalized to housekeeping genes. Suitable oligonucleotide primers are selected by those of skill in the art.

In one embodiment, mass spectroscopy can be used to detect either nucleic acid or protein. Any antibody-based technique for determining a level of expression of a protein of interest can be used. For example, immunoassays such as ELISA, Western blotting, flow cytometry, immunofluorescence, and immunohistochemistry can be used to detect protein in patient samples. Combinations of the above methods, such as those employed in the Luminex® xMAP technology can also be used in the present invention.

Analysis of a protein or nucleic acid can be achieved, for example, by high pressure liquid chromatography (HPLC), alone or in combination with mass spectrometry (e.g., MALDI/MS, MALDI-TOF/MS, tandem MS, etc.).

Analysis of nucleic acid can be achieved using routine techniques such as northern analysis, reverse-transcriptase polymerase chain reaction (RT-PCR), microarrays, sequence analysis, or any other methods based on hybridization to a nucleic acid sequence that is complementary to a portion of the marker coding sequence (e.g., slot blot hybridization) are also within the scope of the present invention. Applicable PCR amplification techniques are described in, e.g., Ausubel et al., Theophilus et al., and Innis et al., supra. General nucleic acid hybridization methods are described in Anderson, “Nucleic Acid Hybridization,” BIOS Scientific Publishers, 1999. Amplification or hybridization of a plurality of nucleic acid sequences (e.g., genomic DNA, mRNA or cDNA) can also be performed from mRNA or cDNA sequences arranged in a microarray. Microarray methods are generally described in Hardiman, “Microarrays Methods and Applications: Nuts & Bolts,” DNA Press, 2003; and Baldi et al., “DNA Microarrays and Gene Expression: From Experiments to Data Analysis and Modeling,” Cambridge University Press, 2002.

Non-limiting examples of sequence analysis include Sanger sequencing, capillary array sequencing, thermal cycle sequencing (Sears et al., Biotechniques, 13:626-633 (1992)), solid-phase sequencing (Zimmerman et al., Methods Mol. Cell Biol., 3:39-42 (1992)), sequencing with mass spectrometry such as matrix-assisted laser desorption/ionization time-of-flight mass spectrometry (MALDI-TOF/MS; Fu et al., Nature Biotech., 16:381-384 (1998)), and sequencing by hybridization (Chee et al., Science, 274:610-614 (1996); Drmanac et al., Science, 260:1649-1652 (1993); Drmanac et al., Nature Biotech., 16:54-58 (1998)). Non-limiting examples of electrophoretic analysis include slab gel electrophoresis such as agarose or polyacrylamide gel electrophoresis, capillary electrophoresis, and denaturing gradient gel electrophoresis.

A detectable moiety can be used in the assays described herein (direct or indirect detection). A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, metals, and the like.

In another embodiment, antibody reagents can be used in assays to detect expression levels of protein biomarkers of the invention in patient samples using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used (see, e.g., Self et al., Curr. Opin. Biotechnol., 7:60-65 (1996)). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (META); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed. Sci., 699:463-80 (1997)). Liposome immunoassays, such as flow-injection liposome immunoassays and liposome immunosensors, are also suitable for use in the present invention (see, e.g., Rongen et al., J. Immunol. Methods, 204:105-133 (1997)). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, CA; Kit #449430) and can be performed using a Behring Nephelometer Analyzer (Fink et al., J. Clin. Chem. Clin. Biochem., 27:261-276 (1989)).

Specific immunological binding of the antibody to a protein can be detected directly or indirectly. Direct labels include fluorescent or luminescent tags, metals, dyes, radionuclides, and the like, attached to the antibody. An antibody labeled with iodine-125 (¹²⁵I) can be used. A chemiluminescence assay using a chemiluminescent antibody specific for the protein marker is suitable for sensitive, non-radioactive detection of protein levels. An antibody labeled with fluorochrome is also suitable. Examples of fluorochromes include, without limitation, DAPI, fluorescein, Hoechst 33258, R-phycocyanin, B-phycoerythrin, R-phycoerythrin, rhodamine, Texas red, and lissamine. Indirect labels include various enzymes well known in the art, such as horseradish peroxidase (HRP), alkaline phosphatase (AP), ÿ-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a ÿ-galactosidase detection system can be used with the chromogenic substrate o-nitrophenyl-ÿ-D-galactopyranoside (ONPG), which yields a soluble product detectable at 410 nm. An urease detection system can be used with a substrate such as urea-bromocresol purple (Sigma Immunochemicals; St. Louis, Mo.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis can be made using a spectrophotometer such as an EMAX Microplate Reader (Molecular Devices; Menlo Park, Calif.) in accordance with the manufacturer's instructions. If desired, the assays of the present invention can be automated or performed robotically, and the signal from multiple samples can be detected simultaneously.

The antibodies can be immobilized onto a variety of solid supports, such as magnetic or chromatographic matrix particles, the surface of an assay plate (e.g., microtiter wells), pieces of a solid substrate material or membrane (e.g., plastic, nylon, paper), and the like. An assay strip can be prepared by coating the antibody or a plurality of antibodies in an array on a solid support. This strip can then be dipped into the test sample and processed quickly through washes and detection steps to generate a measurable signal, such as a colored spot.

Useful physical formats comprise surfaces having a plurality of discrete, addressable locations for the detection of a plurality of different biomarkers. Such formats include protein microarrays, or “protein chips” (see, e.g., Ng et al., J. Cell Mol. Med., 6:329-340 (2002)) and certain capillary devices (see, e.g., U.S. Pat. No. 6,019,944). In these embodiments, each discrete surface location may comprise antibodies to immobilize one or more protein markers for detection at each location. Surfaces may alternatively comprise one or more discrete particles (e.g., microparticles or nanoparticles) immobilized at discrete locations of a surface, where the microparticles comprise antibodies to immobilize one or more protein markers for detection.

The analysis can be carried out in a variety of physical formats. For example, the use of microtiter plates or automation could be used to facilitate the processing of large numbers of test samples. Alternatively, single sample formats could be developed to facilitate prognosis in a timely fashion.

### IV. Compositions, Kits, and Arrays

The present invention provides compositions, kits and integrated systems for practicing the assays described herein using antibodies specific for the polypeptides or nucleic acids specific for the polynucleotides of the invention.

Kits for carrying out the diagnostic assays of the invention typically include a probe that comprises an antibody or nucleic acid sequence that specifically binds to polypeptides or polynucleotides of the invention, and a label for detecting the presence of the probe. The kits may include several antibodies or polynucleotide sequences encoding polypeptides of the invention, e.g., a cocktail of antibodies that recognize the proteins encoded by the biomarkers of the invention.

The invention provides assay compositions for use in solid phase assays; such compositions can include, for example, one or more polynucleotides or polypeptides of the invention immobilized on a solid support, and a labeling reagent. In each case, the assay compositions can also include additional reagents that are desirable for hybridization. Modulators of expression or activity of polynucleotides or polypeptides of the invention can also be included in the assay compositions.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical images.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen are sampled to obtain light intensity readings for each position. Multiple pixels are processed in parallel to increase speed. The apparatus and methods of the invention are easily used for viewing any sample, e.g., by fluorescent or dark field microscopic techniques.

### V. Examples

The following examples are offered to illustrate, but not to limit, the claimed invention.

### Materials and Methods

Human and mouse tissue. Sections were obtained from uninjured C57BL/6 mouse tracheas as well as from C57BL/6 mouse syngeneic tracheal transplants. We used a well-established, reproducible model of tracheal epithelial regeneration using syngeneic subcutaneous tracheal transplants from wild-type C57Bl/6 mice into wild-type C57Bl/6 mice (Jackson Labs, Bar Harbor, Me.) [21, 22]. For this model, donor wild-type C57Bl/6 mice were euthanized and the tracheas dissected out, removing the blood supply to the tracheas and causing an hypoxic-ischemic injury. Recipient wild-type C57Bl/6 mice were sedated with ketamine and an incision was made in the skin of the back of the mice. The donor tracheas were placed heterotopically under the skin of the recipient mice. Mice were euthanized at 7, 14 and 21 days after transplantation and the tracheal transplants were harvested for fixation in formalin and then paraffin embedding. Animal use for these studies was approved by the Department of Laboratory Animal Medicine, David Geffen School of Medicine at UCLA. Tissue sections were obtained from human lung cancer specimens archived in the UCLA Lung Cancer SPORE tissue bank (IRB#02-07-011). The research protocol was approved by the UCLA Institutional Review Board and all human participants gave written informed consent.

Dual immunofluorescence and immunohistochemistry. Dual immunofluorescence was performed as described [18]. Briefly, tracheal tissue was fixed in 4% paraformaldehyde for 18-24 hours and then embedded in paraffin and sectioned. Sections (4 μm) were deparaffinized in xylenes and rehydrated in graded ethanols and boiled in 10 mM sodium citrate buffer for 10 min. Blocking was performed with serum-free protein block (Dakocytomation). The primary antibodies used were rabbit anti-mouse K5 (dilution 1:500; Abcam, Cambridge, Mass.), mouse anti-K14 (dilution 1:20; Abcam) and rabbit polyclonal anti-PCNA (Dilution 1:50; Abcam).

For calculating the proportion of proliferating (PCNA+), K5+, and K14+ cells in the epithelia, premalignant lesions, or tumors, tissue immunofluorescence images were obtained using a Zeiss Axiolmager microscope (Carl Zeiss, Germany). For mouse samples, cross sections through the same level of each trachea were selected for measurement. Cells were manually counted at 20× magnification. Total K14+K5+ cells and K14-K5+ cells in an epithelium or lesion were counted to determine the percentage of K14+ cells within all the K5-expressing cells. K14+PCNA+ cells and K14+PCNA− cells in a premalignant lesion or tumor lesion were counted to determine the percentage of K14-expressing cells that were proliferating.

Immunohistochemical analysis of human lung tissue was performed as described [19] with the K5 and K14 antibodies described above. The lung TMAs were sectioned just prior to use, and serial sections were stained for K14 or K5 using a two-step immunohistochemical protocol.

Histological definitions: Reserve cell hyperplasia was defined as a continuous and double layer of basal cells. Squamous metaplasia requires development of horizontally oriented squamous cells with intercellular bridges. Dysplasia was diagnosed in the setting of epithelial thickening with nuclear pleomorphism and partial loss of normal maturation from the basal to luminal surface. Carcinoma in situ has marked nuclear pleomorphism and coarse chromatin with no maturation from basal to luminal surface and the absence of frank invasion.

Lung cancer tissue microarray (TMA). The TMAs were constructed under appropriate IRB and HIPAA regulations using formalin-fixed, paraffin-embedded archival lung samples from the UCLA Department of Pathology and Laboratory Medicine and the lung cancer Specialized Program of Research Excellence (SPORE) tissue bank at The University of Texas M. D. Anderson Cancer Center (Houston, Tex.) [19]. The characteristics of these TMAs have been previously described in detail [19]. The TMA was scored in a semi-quantitative fashion by a pathologist (MA), and spot-checked by a second pathologist (VM), both of whom were blinded to clinical and outcomes information. K5 and K14 cytoplasmic staining was quantified based on the intensity and frequency of cell staining, similar to previously described methods [19]. A total of 399 patients from the UCLA TMA and 505 patients from the M.D. Anderson TMA were used in these studies.

Statistical analysis. Analyses were performed using the open source R software (http://www.R-project.org) including survival, Design and Hmisc packages. Pooling criteria were similar to those previously described [19]. K5 and K14 expression differences among various subgroups were determined using the Wilcoxon signed rank test or Kruskal-Wallis rank sum test. For dichotomized (positive versus negative staining for K5 and K14) expression, the Fisher exact test was used for analysis with categorical variables such as stage, grade, smoking history and presence of metastasis. Survival curves were calculated using the Kaplan-Meier method and comparisons were made using the log-rank test. The Cox proportional hazards model (univariate and multivariate) was used to determine the significance of various factors related to survival. LogRank and Fisher exact P-values were two-sided and a P<0.05 was considered significant.

Quantitative Real-Time PCR. Two 7 μm sections of frozen tissues were scraped from serial sections of lung tumors and total RNA was isolated using the Trizol (Invitrogen, Carlsbad, Calif.) protocol. Quantitative real-time PCR for K14 and GAPDH was performed as described using primers and probe from Applied Biosystems (Carlsbad, Calif.) [20]. Cycling conditions used were 20 sec at 95° C., and 40 cycles of 1 sec denaturation at 95° C. and 20 sec annealing at 60° C. The triplicate Ct values for each sample were averaged resulting in mean Ct values for both K14 and GAPDH. Fold change was calculated by using the 2(-ΔΔCt) formula.

Western Blot Analysis. Cell lysates were extracted from frozen sections and resolved on a 12% SDS-polyacrylamide gel, followed by transfer to nitrocellulose membranes (Bio-Rad, Richmond, Calif.) Immunoblotting was performed as previously described [8]. The primary antibody used was rabbit anti-K14 (1:200; Abcam), followed by secondary goat anti-rabbit HRP conjugated antibody (1:3000; Bio-Rad). The immunocomplexes were visualized using SuperSignal West Pico Chemiluminescent System (Thermo Scientific, Rockford, Ill.).

Lung cancer microarray expression data sets and analysis. A search of both the Gene Expression Omnibus (GEO) and the scientific literature was performed for data sets in which microarray profiles of lung adenocarcinomas and squamous cell carcinomas and the associated survival data were freely available. For those data sets for which raw Affymetrix CEL files were available, the data were re-normalized using both the Robust Multiarray Average (RMA) and the MicroArray Suite 5.0 (MAS5) algorithms as well as an Entrez Gene ID probeset mapping (http://brainarray.mbni.med.umich edu/Brainarray/Database/CustomCDF). In these data sets, arrays were removed prior to analysis if the MAS5 Percent Present quality metric was more than 2 standard deviations below the mean or if the Scaling Factor was greater than 3. For samples from the Bhattacharjee data set that were run on replicate arrays, the K14 expression levels were averaged, and the MAS5 call of the result was Present if K14 was called Preseent in both replicates. For all other data sets, the complete set of preprocessed data available from GEO was used.

An iterative approach was used to determine which samples could be considered K14 positive or K14 negative within each data set for categorical analysis. At each iteration, the distribution of K14 expression values in a data set was tested for normality using the Shapiro-Wilk test. If the distribution was significantly different from normal (p<0.05), the highest value was removed. This process was repeated until the distribution of the set X of remaining values was not significantly different from a normal distribution. The entire set of K14 expression values for that data set was then z-score normalized to X by subtracting the mean of X from each value and then dividing the result by the standard deviation of X. Any normalized values greater than 3 were considered K14 positive. Furthermore, any K14 expression values that were not called Present by MAS5 (in those data sets for which CEL files were available) were automatically considered K14 negative.

Survival analysis was performed using a Cox proportional hazards model. The model was stratified by data set to account for site-specific differences in patient treatment, and survival data was censored at 120 months. Those data sets for which the entire distribution of normalized K14 expression values was significantly (p<0.05) different from a standard normal distribution by Kolmogorov-Smirnov test were considered the “best” sets, and stratified Cox proportional hazards analysis was also performed separately on these sets. All analyses were performed using R 2.9.2.

Bild et al. data set: GEO (GSE3141). Links for the raw CEL files:
Main page:
http://data.cgt.duke.edu/oncogene.php

### Files:

https://discovery.genome.duke.edu/express/resources/1136/LungtumorData.zip
https://discovery.genome.duke.edu/express/resources/1136/Lung_clinical_summary.xls
Bhattacharjee et al. data set:
http://www.broadinstitute.org/mpr/lung/http://www.broadinstitute.org/mpr/publications/projects/LUNG/datasetAscans.txt
http://www.pnas.org/content/suppl/2001/11/13/191502998.DC1/SampleData.xls

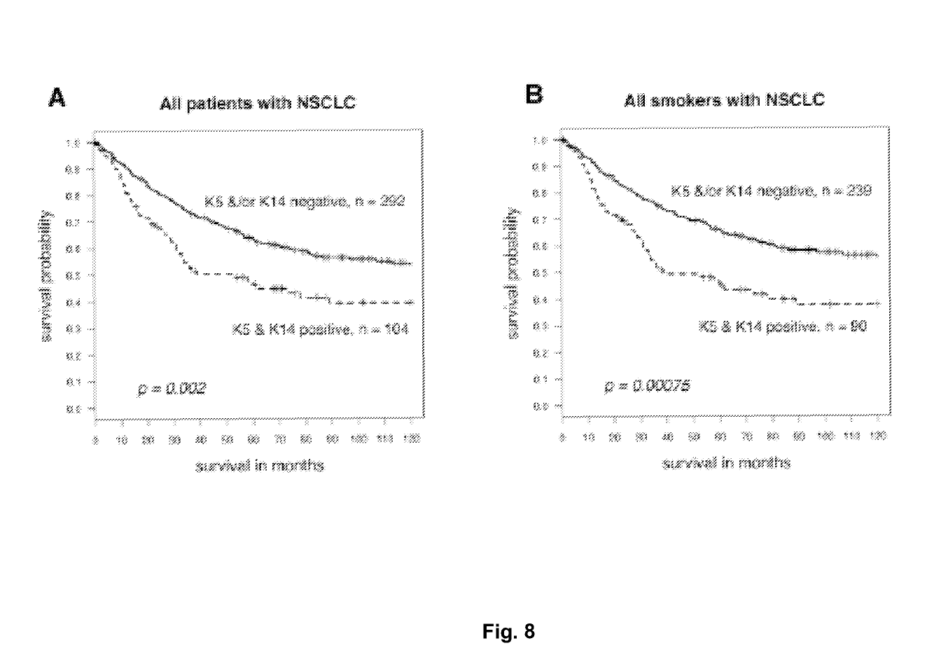

Robustness of TMA dichotomization. Using log rank p-value and hazard ratios from the Cox regression model, cutpoints were checked from 1% to 99% cells positive at intervals of 1%. For all patients together, significant results for survival difference were obtained in the range of 3-28% (except for the cutpoint at 21% where p=0.06). The 5% cutpoint was chosen as a relatively rounded number that could possibly be used in a clinical diagnostic setting. In addition, we analyzed the TMA for K14 and K5 expression and found almost identical results for NSCLC tumors expressing both K5 and K14 to that of K14 expression alone with a worse prognosis in patients with K14+K5+-expressing tumors compared to K14 and/or K5 negative tumors (P=0.002) (FIG. 8).

siRNA Preparation and Transfection. For RNA interference assays, 19-nucleotide small interfering RNA duplex (siRNA) for human keratin 14 (GenBank accession number NM_{—}000526) was used (Ambion, Austin, Tex.). The sequences were as follows: sense, GAGUUGAACCUGCGCAUGAtt (SEQ ID NO: 1); antisense, UCAUGCGCAGGUUCAACUCtg (SEQ ID NO: 2). Silencer FAM-labeled negative control #1 siRNA (Ambion) was used as a control. Cells were transfected with 50 nM siRNA using Lipofectamine 2000 (Invitrogen) transfection reagent according to the manufacturer's recommendations. The cells were harvested 5 days later and analyzed by Western blots.

Cell Proliferation Assay. BEAS2B immortalized normal human bronchial epithelial cells (ATCC, Manassas, Va.) were transfected with control siRNA or K14 siRNA, as described above. Three days after transfection, the cells were plated at different densities in 96-well plates and incubated at 37° C. for a further two days. The CellTiter 96 Non-Radioactive Colorimetric based-Cell Proliferation Assay (Promega) was performed by adding 20 μl of cells in culture medium according to the manufacturer's recommendations. Cells were incubated for 1-4 hr at 37° C. Absorbance was measured spectrophotometrically at a wavelength of 490 nm.

### Results

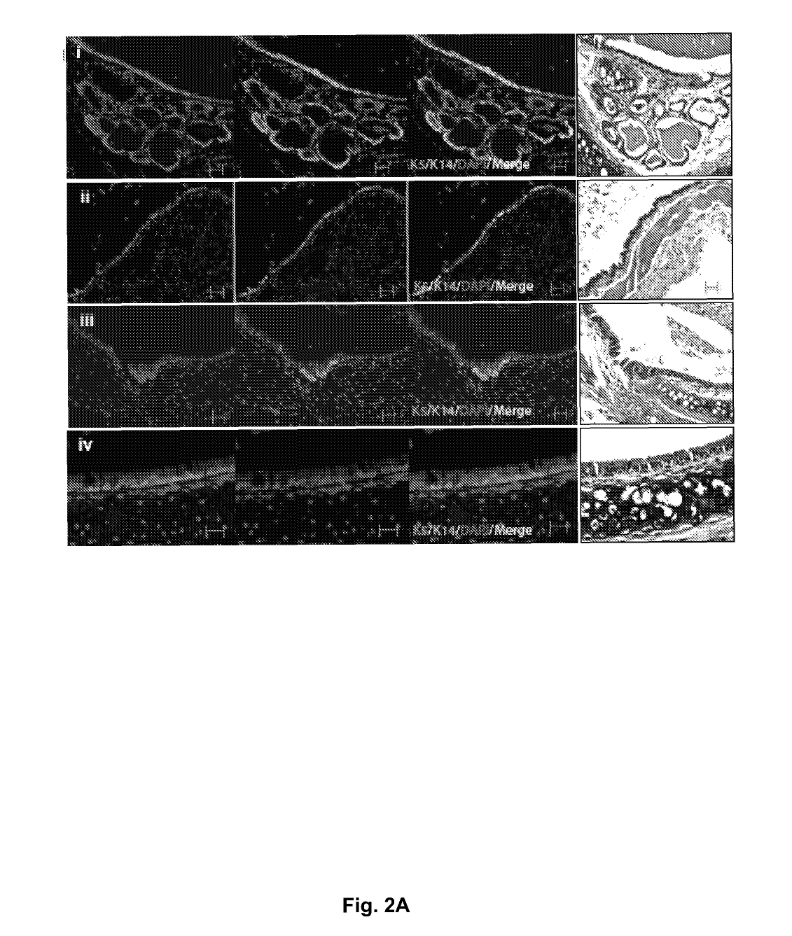

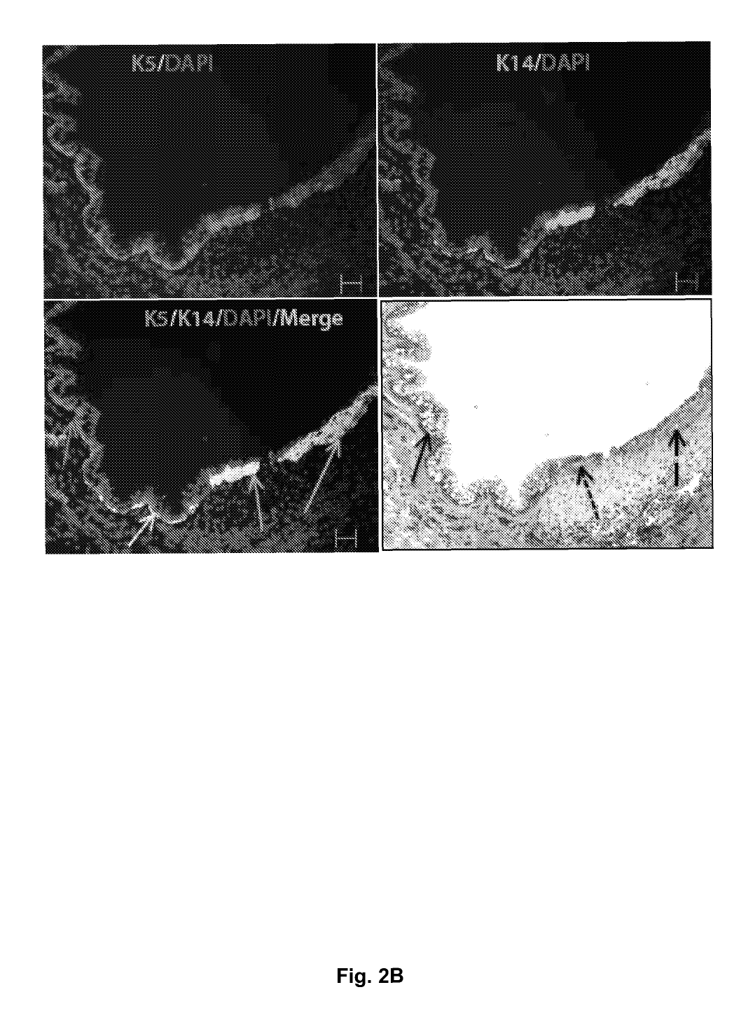

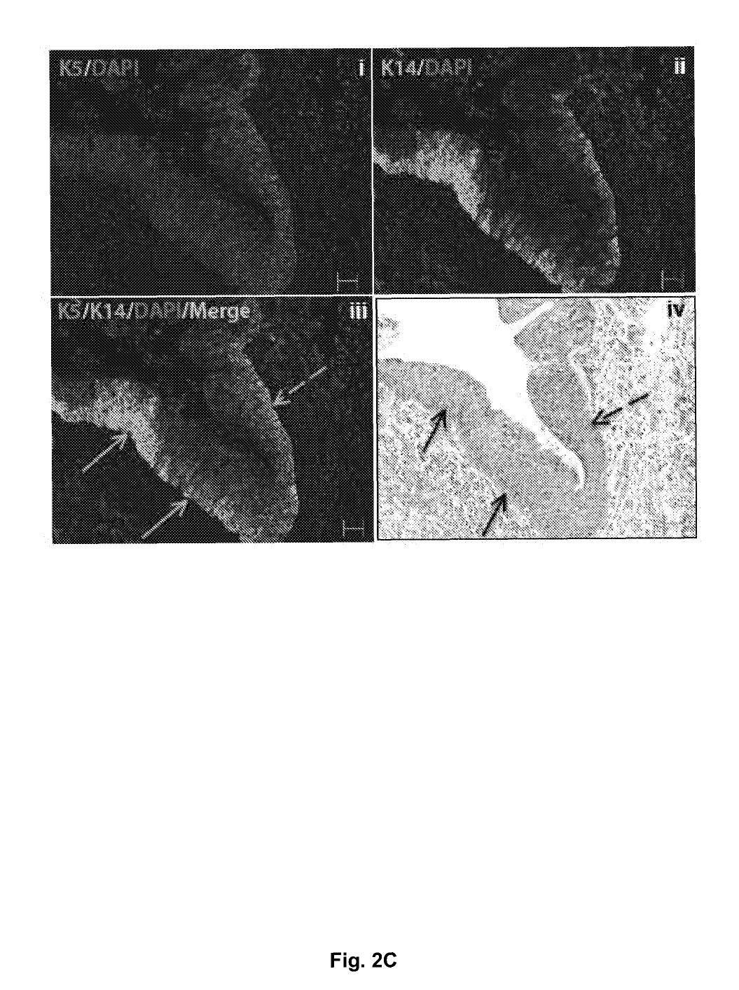

Identification of K14+K5+, K14-K5+ cell populations in the steady state airway epithelium and submucosal glands. We further examined the relative abundance and location of K14+K5+ cells in a model of airway epithelial injury. To do this, we performed heterotopic, syngeneic tracheal transplants in mice and examined the repairing tracheal airways for K14 and K5 expression after hypoxic-ischemic injury [20,21]. We found K14+K5+ cells in the submucosal glands, submucosal gland ducts, as well as in cells on the basement membrane repairing the surface airway epithelium. These K14+K5+ cells persisted in the airway epithelium during all stages of repair and represented 85.6%±5.3% of all cells of the mouse repairing surface airway epithelium (FIG. 2A, Table 1). In the repaired pseudostratified columnar epithelium only K5 expression was present in the basal cells (FIG. 2A iii, iv).

TABLE 1
Quantification of K5+K14+ cells during airway epithelial repair

| | Mean | Mean | |
|---|---|---|---|
| | Percentage ± | Percentage ± | |
| | SEM (n) | SEM (n) | |
| | Steady state | Repairing airway/ | |
| | airway epithelium | premalignant | P-value |
| Mouse | 10.7 ± 3.4 (6) | 85.6 ± 5.3 (10) | <0.0001 |
| Human | 1.3 ± 0.8 (14) | 75.3 ± 3.4 (12) | <0.0001 |

K14+K5+ cells populate pre-neoplastic and neoplastic lesions. We further explored the expression of K14+K5+ cells in human disease representing chronic injury and repair after smoking. For this we performed dual immunofluorescent staining of airway tissue from patients with chronic obstructive pulmonary disease (COPD). As in the mouse airway injury model, we observed a persistence of K14+K5+ cells in repairing areas of reserve cell hyperplasia (FIG. 2B). We also observed a predominance of K14+K5+ cells in potentially pre-neoplastic lesions represented by squamous metaplasia, dysplasia and carcinoma in situ (FIGS. 2B-C). We found K14+K5+ cells in all premalignant lesions from all patients examined to date and in the premalignant lesions K14+K5+ cells represented 75.3%±3.4% of cells in the lesions (FIGS. 2B-C, Table 1).

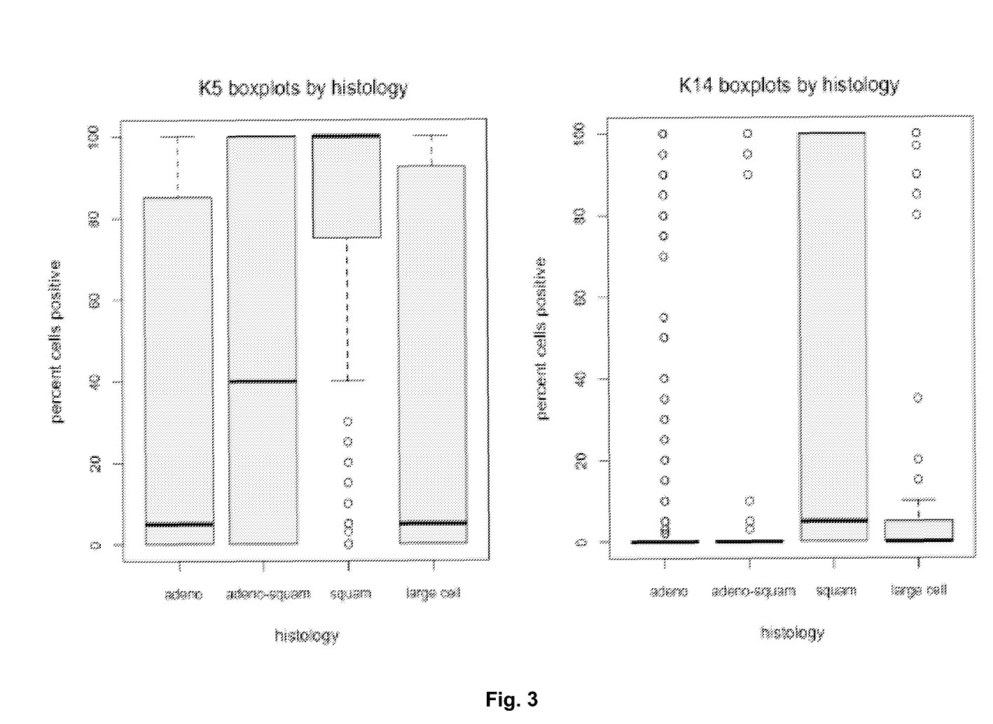

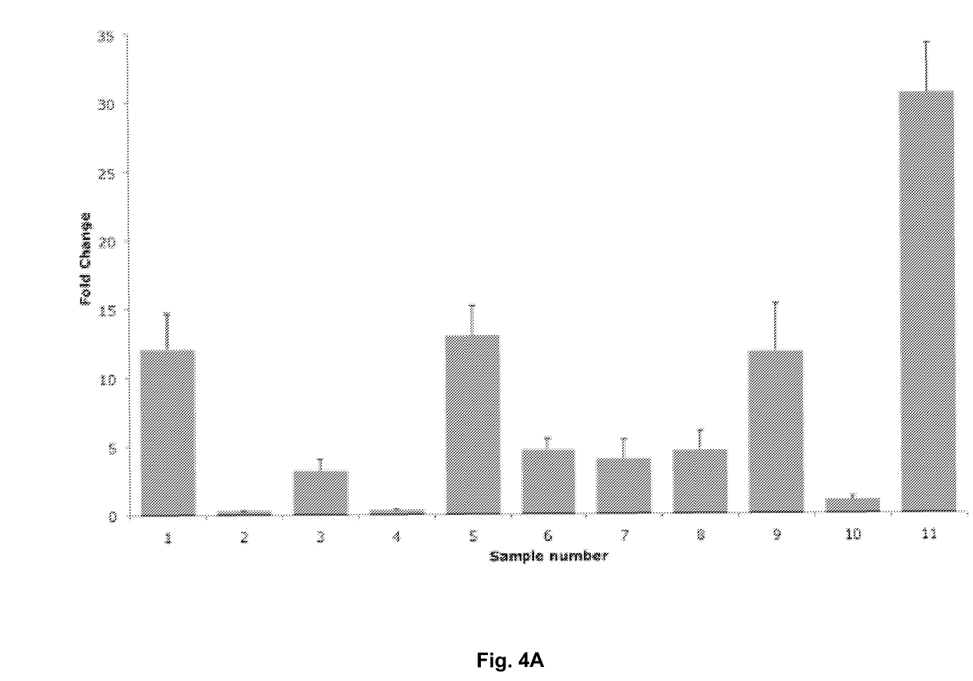

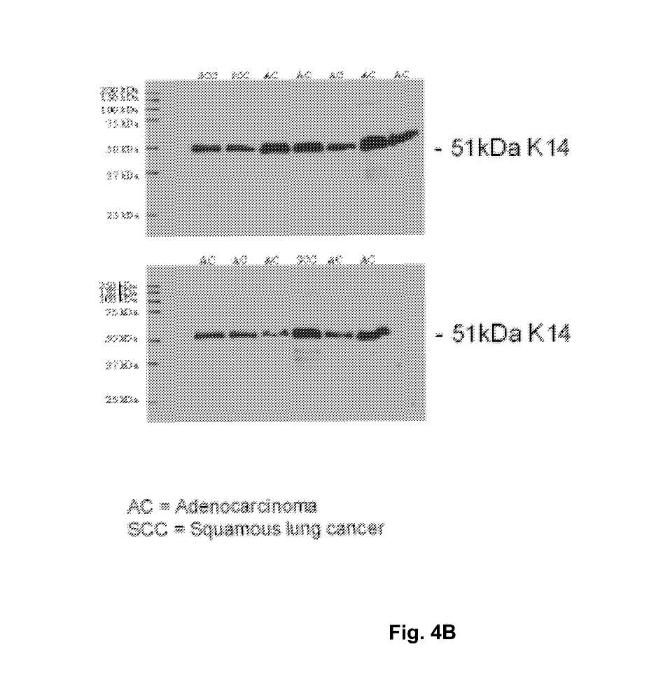

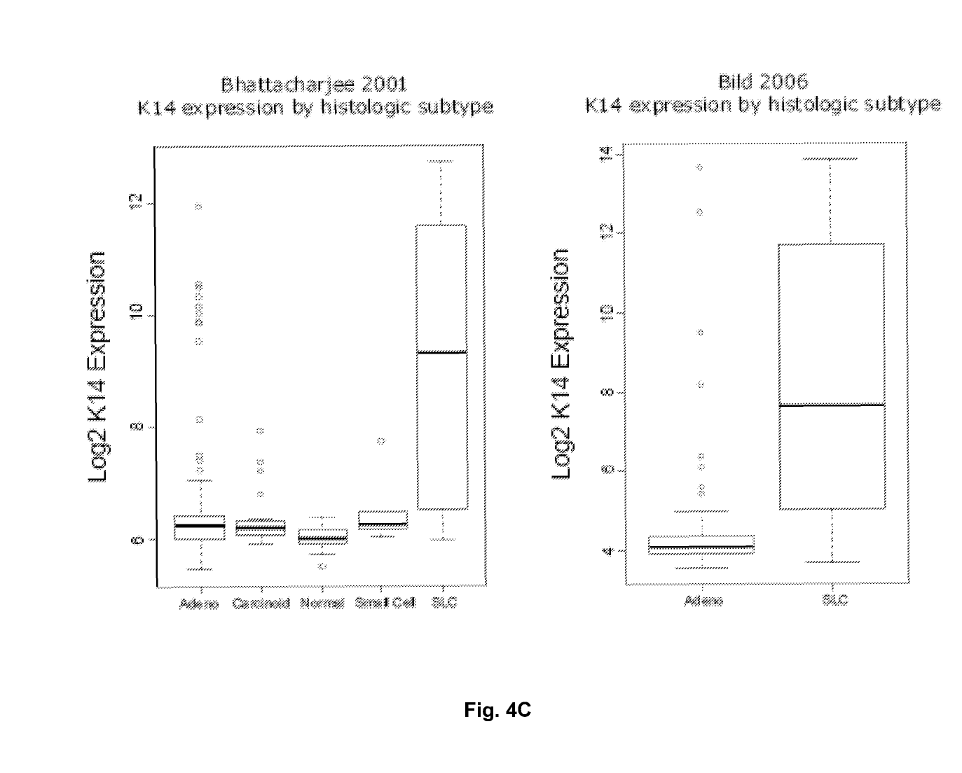

The presence of K14+ cells in NSCLC tumor samples confers a worse prognosis. Based on the over-representation of K5+K14+ cells in pre-neoplastic lesions, we further assessed whether the presence of K14 in primary NSCLC tumors was associated with lung cancer development and/or progression. To do this, we examined protein expression on a population basis using high-density lung TMAs. We first examined 399 patients from the UCLA TMA (adenocarcinoma 237, adenosquamous 19, squamous cell carcinoma 100, neuroendocrine 7, large cell carcinoma 32, other 4). Levels of K14 and K5 were found to be similar in all NSCLC with the notable exception of tumors with squamous differentiation. In squamous cell carcinoma 90% of cells were K5 positive and 60% were K14 positive compared with 57% and 18% of cells in adenocarcinomas respectively (positivity defined by 5% cut point, FIG. 3). These results were verified by quantitative real-time PCR) (FIG. 4A), review of publicly available lung cancer microarray expression data sets (FIG. 4B, Table 2), and Western blot analysis on frozen adenocarcinoma and squamous lung cancer samples (FIG. 4C) [22, 23]. The percentage of K14+ or K5+ cells in NSCLC did not correlate with stage, and although lower grade tumors tended to have somewhat higher percentages of K5 and/or K14 positive cells, a significant association was only seen for K14 in squamous carcinomas (data not shown). Tumor samples from male subjects had slightly higher percentages of both K5+ and K14+ cells than did samples from female subjects (Table 3).

TABLE 2
Publicly available datasets of lung adenocarcinoma samples
with K14-expressing tumors

| | | Total | | | | |
|---|---|---|---|---|---|---|
| | Number | num- | | | Low- | Up- |
| | K14- | ber | | | er | per |
| | positive | sam- | P- | Odds | 95% | 95% |
| Set | samples | ples | value | ratio | CI | CI |
| Bhattacharjee_2001 | 4 | 113 | 0.80 | 0.83 | 0.20 | 3.40 |
| Bild_2006 | 4 | 54 | 0.32 | 1.85 | 0.56 | 6.14 |
| GSE11969 | 4 | 90 | 0.16 | 2.36 | 0.72 | 7.70 |
| GSE8894 | 11 | 62 | 0.53 | 1.31 | 0.56 | 3.03 |
| Shedden_2008_DFCI_1 | 3 | 49 | 0.77 | 0.74 | 0.10 | 5.55 |
| Shedden_2008_DFCI_2 | 3 | 28 | 0.46 | 0.46 | 0.06 | 3.59 |
| Shedden_2008_Moffitt | 5 | 77 | 0.63 | 0.78 | 0.28 | 2.16 |
| Shedden_2008_MSKCC | 4 | 100 | 0.21 | 2.54 | 0.60 | 10.77 |
| Shedden_2008_UMich | 16 | 171 | 0.05 | 1.85 | 1.00 | 3.39 |
| All adenocarcinoma | 54 | 744 | 0.11 | 1.33 | 0.93 | 1.90 |

TABLE 3
Mean percentage K5+ and K14+ cells by subgroup

| | Mean Percentage | | Mean Percentage | |
|---|---|---|---|---|
| Group | K5+ Cells (n) | P-value | K14+ Cells (n) | P-value |
| Stage | | 0.351^{§} | | 0.098^{§} |
| stage I | 46.1 ± 2.8 (229) | | 15.2 ± 2 (222) | |
| stage II | 46 ± 4.9 (72) | | 21 ± 4.1 (71) | |
| stage III | 53.8 ± 4.6 (80) | | 21.1 ± 3.7 (77) | |
| stage IV | 42.9 ± 8.4 (27) | | 16.7 ± 5.5 (27) | |
| Grade | | 0.027^{§} | | 0.292^{§} |
| grade 1 | 48.7 ± 5.1 (66) | | 18.4 ± 4.2 (65) | |
| grade 2 | 56.8 ± 3.9 (114) | | 21.5 ± 3.3 (108) | |
| grade 3 | 44.5 ± 3.2 (163) | | 17.1 ± 2.4 (161) | |
| grade 4 | 36.8 ± 7.1 (35) | | 13.4 ± 4.7 (34) | |
| Gender | | 0.035* | | 0.026* |
| women | 44.1 ± 2.8 (211) | | 14.3 ± 2 (206) | |
| men | 50.6 ± 3.1 (198) | | 20.7 ± 2.4 (192) | |

*Mann-Whitney U test
^{§}Kruskal-Wallis test

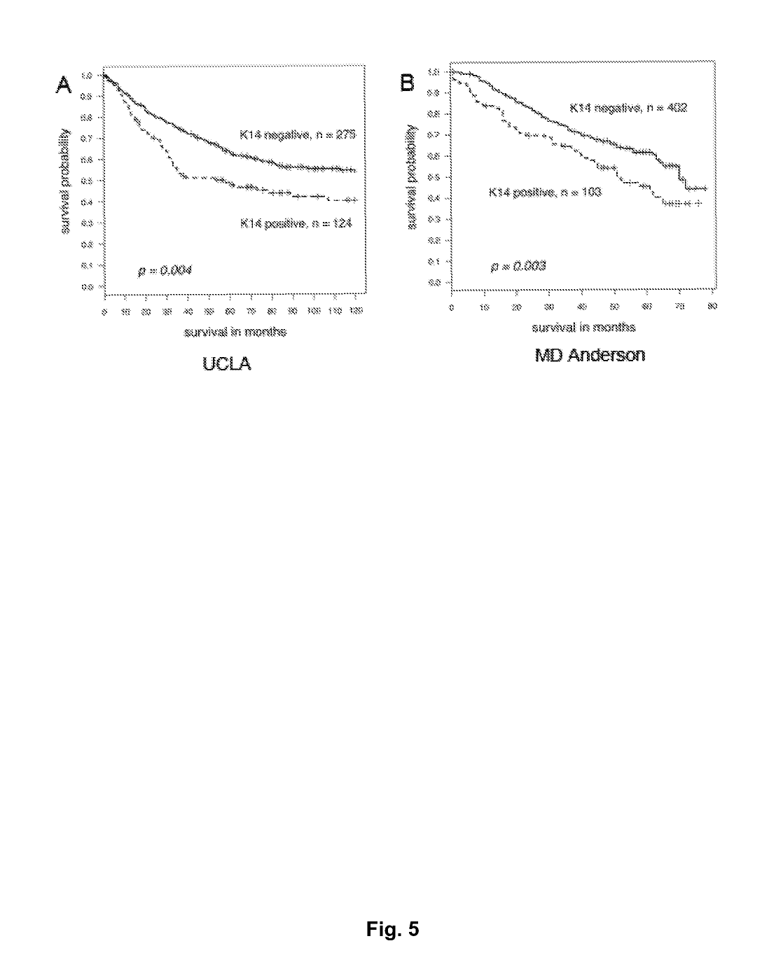

We further examined whether tumors expressing K14 represented a more aggressive substratum of tumors. Consistent with this, patients with NSCLC that expressed K14 were found to have a significantly worse prognosis than patients with NSCLC in which K14 was below the level of detection (P=0.004, hazard ratio=1.58) (FIG. 5A). We also validated this TMA data with an independent TMA obtained from the M.D. Anderson Cancer Center. We found identical results to those found on the UCLA TMA: patients with K14-expressing tumors had a worse prognosis (p=0.003, hazard ration=1.60) (FIG. 5B).

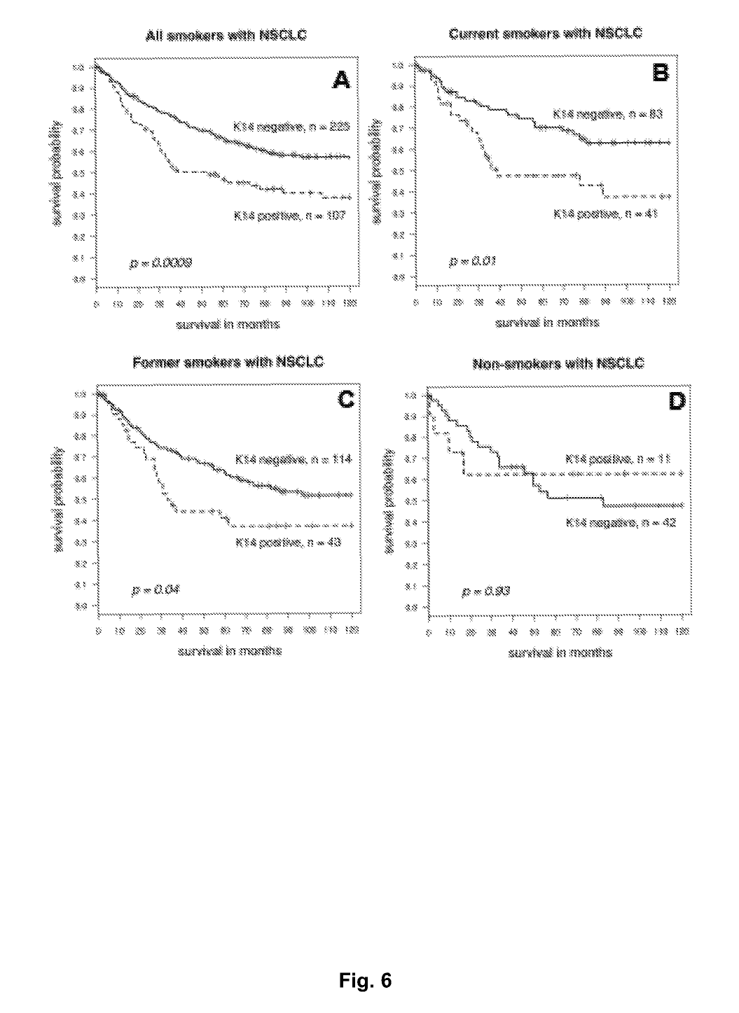

The presence of K14+ cells in NSCL tumor samples confers a worse prognosis in smokers and is associated with metastasis. It is generally accepted that cigarette smoking has a causal relationship with lung cancer. Smoking results in chronic airway epithelial injury and dysfunctional repair is commonly seen [24]. We hypothesized that the presence of dysregulated K14+ reparative cells that predict poor prognosis might have resulted from chronic smoking injury. Consistent with this hypothesis, we found a striking increase in the predictive value of K14 expressing NSCLC tumors in individuals who were current or former smokers (P=0.001, hazard ratio=1.77) (FIG. 6A). In all smokers, K14 positivity (>5%) was an independent predictor of poor prognosis (P=0.027) in a multivariate Cox proportional hazards model, which also included stage, grade, and age (Table 4). When separating current from former smokers, the predictive value of K14 positivity was more pronounced in current smokers (P=0.01, hazard ratio=2.11, FIG. 6B). Current smokers were defined as those patients who were currently smoking or who had quit within one year of when their tissue sample was collected. However, K14 positivity was still predictive of poor prognosis in former smokers as well (P=0.04; hazard ratio=1.68; FIG. 6C). Former smokers were defined as those patients who had quit more than a year before their tissue sample was collected. This was true for individuals with either squamous cell carcinoma or adenocarcinomas. In never smokers, the presence of K14+ cells had no predictive value for outcome (FIG. 6D). Never smokers were defined as having smoked less than 100 cigarettes over their lifetime.

TABLE 4
Multivariate Cox proportional hazards analysis for all smokers
(n = 308)

| | Hazard ratio | |
|---|---|---|
| | (95% confidence | |
| Variable | interval) | P-value |
| K14 positivity | 1.49 (1.05-2.13) | 2.67E−02 |
| tumor stage | 1.90 (1.62-2.23) | 5.11E−15 |
| tumor grade | 1.21 (0.98-1.49) | 8.05E−02 |
| age | 1.04 (1.02-1.06) | 2.31E−04 |

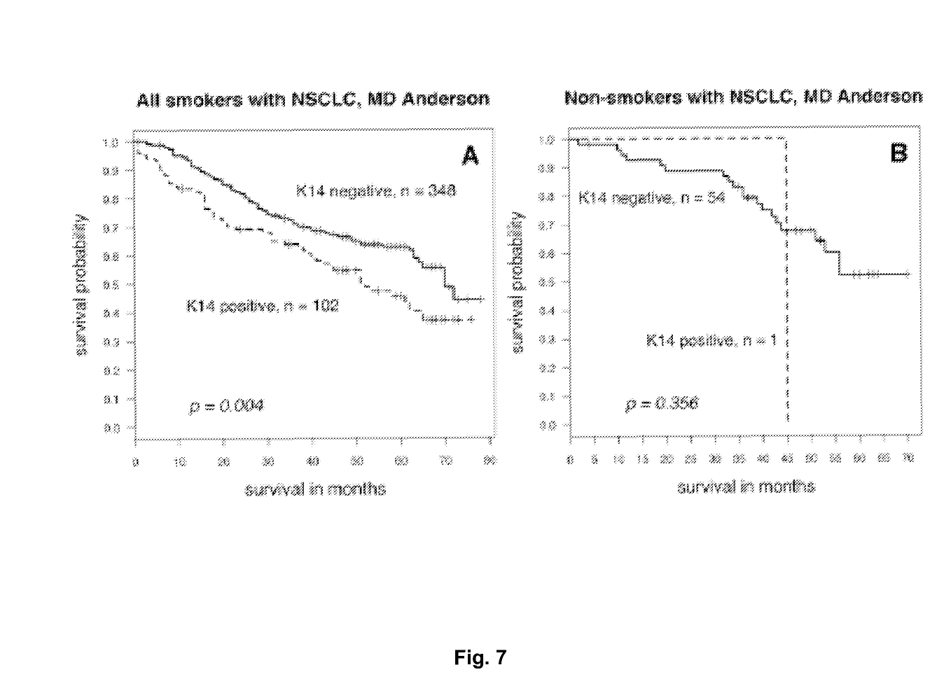

Validation of these results was performed on an independent TMA from the M.D. Anderson Cancer Center and smoking was again associated with poor prognosis (P=0.004, hazard ration=1.59) (FIG. 7A), but again there was no association between K14 expression and prognosis in non-smokers (P=0.356, hazard ratio=2.51) (FIG. 7B).

We analyzed K5+K14+ cells in the TMA analysis and found almost identical results to that of K14 expression alone. The Kaplan Meier analysis revealed a significantly worse prognosis in NSCLC patients with K5+K14+ tumors (P=0.002), and again this was especially significant in smokers (P=0.00075). These survival curves are included in FIG. 8.

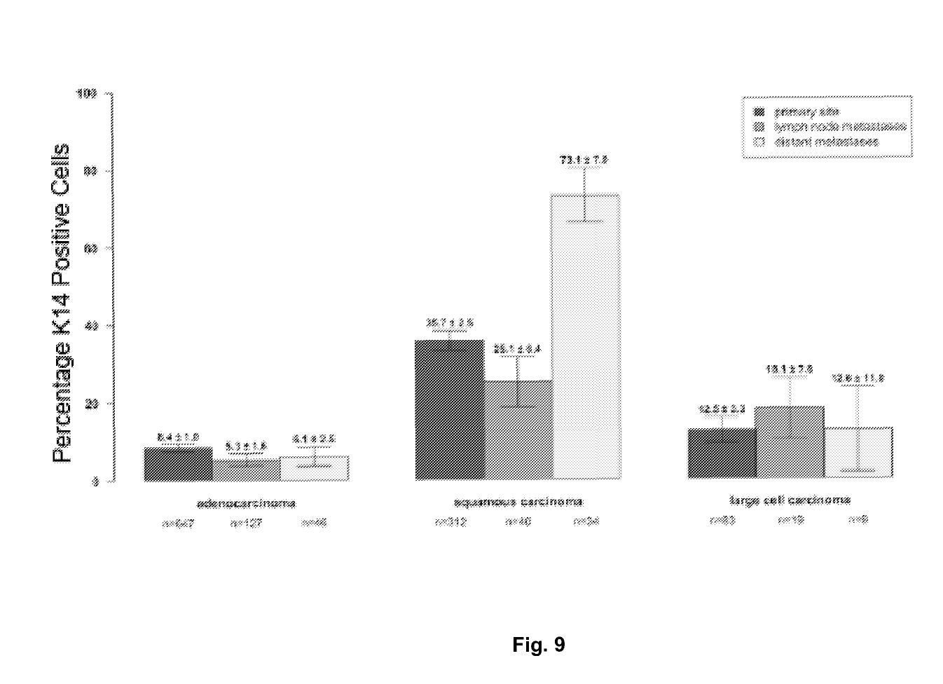

We found that the presence of K14+ cells in the primary tumors of current smokers was associated with metastatic disease (P=0.02) (Table 5). We further found that non-adenocarcinoma primary NSCLCs from smokers with metastases had a higher percentage of K14+ cells. (P=0.004) (Table 6). Examination of K14 expression in distant metastatic sites revealed a significant increase in the number of K14+ cells in metastases compared to the primary sites in squamous lung cancer (P<0.001), but not in other histologic subtypes (FIG. 9).

TABLE 5
Primary tumor K14 presence/absence in current smokers: no
metastases vs. any metastases

| Histology (n) | Fisher P-value |
|---|---|
| All histologies (124) | 0.02 |
| Adenocarcinoma (61) | 0.71 |
| Squamous carcinoma (39) | 0.05 |
| Large cell carcinoma (14) | 0.09 |
| Not adenocarcinoma (63) | 0.004 |

TABLE 6
Mean percentage K14+ cells in current smokers: no
metastases vs. any metastases

| | Mean | Mean | |
|---|---|---|---|
| | percentage | percentage | Mann- |
| | K14+ cells, | K14+ cells, | Whitney |
| Histology | no mets (n) | any mets (n) | P-value |
| All histologies | 12.7 (84) | 26.9 (40) | 0.033 |
| Adenocarcinoma | 7.7 (44) | 4.5 (17) | 0.424 |
| Squamous carcinoma | 21.8 (24) | 53.4 (15) | 0.023 |
| Large cell carcinoma | 11.6 (9) | 31.5 (5) | 0.061 |
| Not adenocarcinoma | 18.2 (40) | 43.4 (23) | 0.004 |

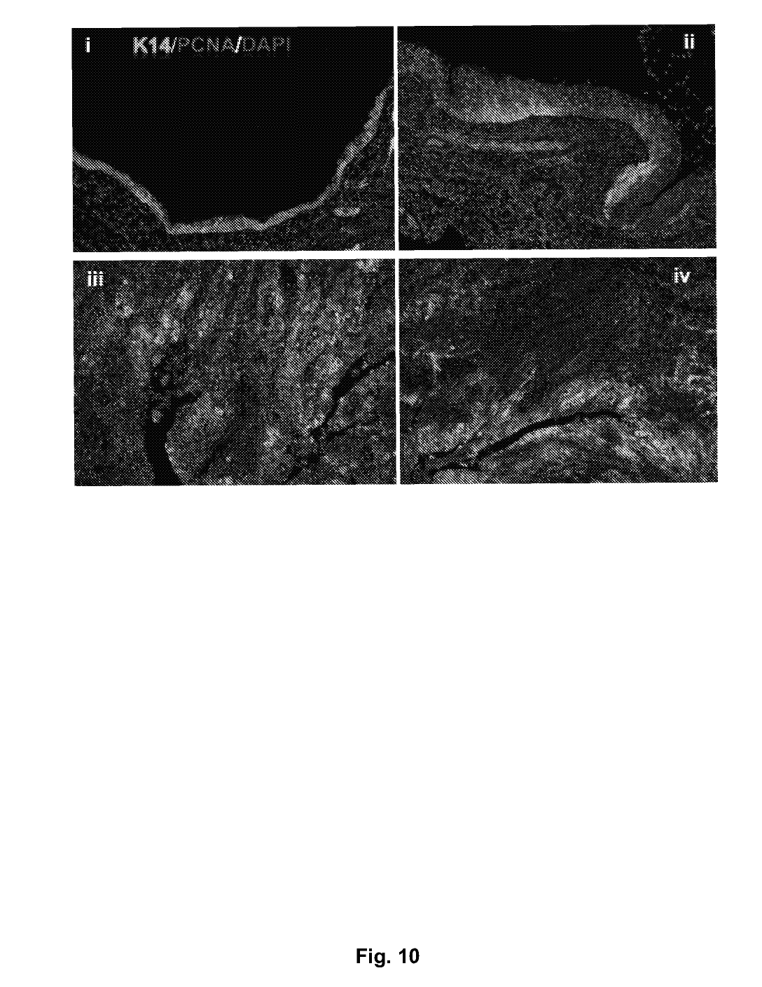

K14 expression is not a marker of proliferation. We next assessed whether K14 expression was prognostic merely because it might be a surrogate marker for cell proliferation. Therefore, in order to determine whether the poor prognosis in K14-expressing tumors was related to increased proliferation in these tumors, we performed dual immunostaining for K14 and PCNA to assess the percentage of K14-expressing cells that are also proliferating in premalignant lesions and NSCLC. In premalignant lesions we found that 57.8%±5.1% of K14+ cells also expressed PCNA (FIG. 10i, ii). In squamous lung cancer patient samples, we found that 67.3%±7.3% of K14+ cells also expressed PCNA (FIG. 10iii, iv). We also found many other cell populations, which were K14 negative that expressed PCNA. There was also clearly a subpopulation of K14+ cells that were not proliferating (FIG. 10). K14-expressing cells are therefore not a unique marker of proliferating cells, as many other cell populations are proliferating in lung cancer. This is consistent with the point that K14 is a marker of poor prognosis but may not functionally be important for proliferation.

In addition, we performed K14 knockdown studies in BEAS2B immortalized normal human bronchial epithelial cells. We used siRNA technology to reduce expression of K14 in BEAS2B cells by 90% at 5 days post-transfection compared to control siRNA transfected cells. The MTS proliferation assay showed no effect on cell proliferation in the K14 siRNA transfected cells compared to the control siRNA transfected cells and there was also no effect on cell morphology. Similarly, PCNA expression in transfected cells was found to be equivalent by western blot analysis in K14 siRNA and control siRNA transfected cells (FIG. 11).

### Discussion

Cigarette smoking causes cycles of injury and repair of the airway and is a known cause of lung cancer [24]. We, and others, have shown that K14+ progenitor cells are a reparative cell population and contribute to repair of the epithelium of the cartilaginous airways and in the more distant bronchioles after injury, such as hypoxic-ischemic injury, naphthalene injection and sulfur dioxide inhalation [12, 16]. Here we propose that in the context of injury, K5+K14+ cells originate from the submucosal gland K5+K14+ cells and/or from the K5+K14− basal cells that then acquire K14 expression on the repairing surface airway epithelium. However, once normal repair is completed, K14 expression is no longer seen in the mature basal cells of the pseudostratified columnar epithelium. This implies that K14 expression is tightly regulated at steady state and the persistence of K5+K14+ cells on the surface airway epithelium after injury represents self-renewing cells that do not differentiate to mature airway epithelial cell types and represent dysregulated repair. Our data are, therefore, consistent with the development of dysregulated repair after injury leading to a self-renewing K14+ progenitor cell population in premalignant lesions. These cells could therefore potentially survive long enough to accumulate the genetic and epigenetic mutations that are thought to be necessary to develop a tumor [3].

We have found that the presence of dysregulated K14+ progenitor cells in NSCLC after chronic smoking injury is associated with increased mortality from lung cancer. This implies that there could be a novel putative tumor-initiating cell population in a subset of smoking-related NSCLCs with a poor prognosis. In mice, a putative lung stem call was isolated, termed the bronchoalveolar stem cell (BASC), which expressed markers of both Clara cells (CCSP) and type II pneumocytes (SP-C), proliferated for repair, and which was seen in the earliest cancerous lesions and increased as the tumors advanced [25]. However, it is not clear what the equivalent human cell surface markers are that would enable the purification and propagation of these cells in xenograft models in order to determine whether these cells are CSCs in lung cancer patients. In addition, the heterogeneity of lung cancers suggests that there are likely to be multiple tumor-initiating cell populations for different lung cancer histologic subtypes and locations. K14-expressing cells have been found for repair in the distal bronchioles [16] and we found K14 mRNA and protein expression in adenocarcinomas as well as squamous cell cancers. In addition, K14 expression correlated with poor prognosis in all NSCLC histologic subtypes, although it only correlated with metastases in non-adenocarcinoma histologies.

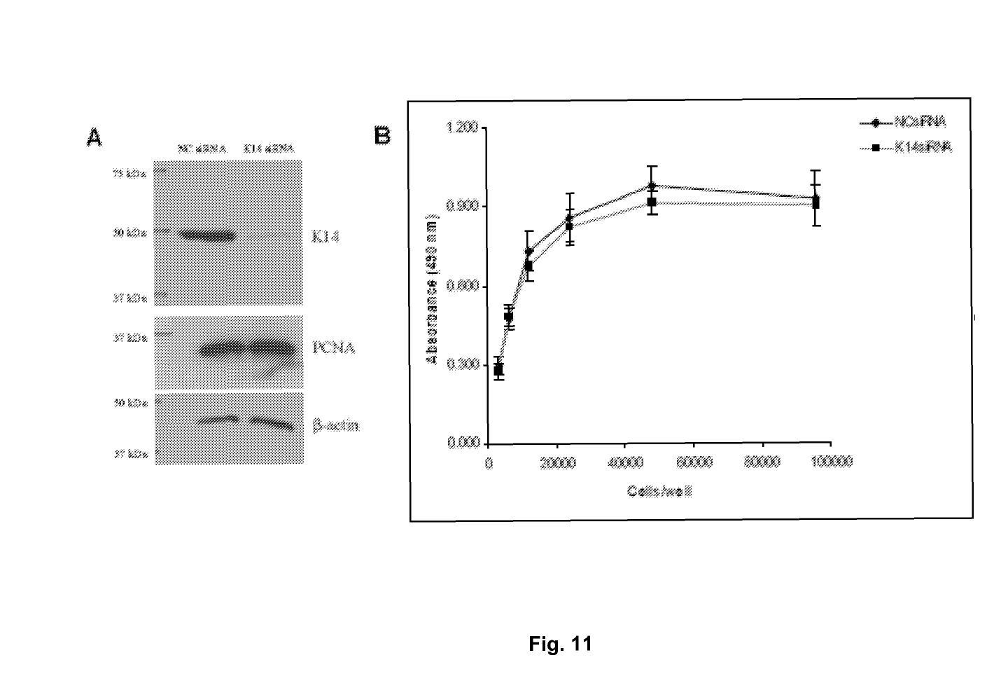

Precursor lesions of squamous lung cancer are known to have high levels of K14 expression, from basal/reserve cell hyperplasia to squamous metaplasia and dysplasia to carcinoma in situ as well as invasive carcinoma itself [28]. Our data suggest that K14-expressing cells in the airway epithelium in premalignant lesions may represent self-renewing, reparative progenitor cells, that may have the potential to be tumor-initiating cells. We also believe that K14 expression alone is not sufficient to generate a malignancy and that subsequent genetic and epigenetic changes are needed to develop NSCLC. This is illustrated by work from Dakir et al who used a mouse Clara cell specific 10 kDa protein promoter (CC10) to constitutively express human K14 in bronchial epithelium. The CC10-hK14 overexpressing transgenic mouse developed a squamous differentiation program in the mouse lung, but failed to promote squamous maturation with rare squamous metaplastic lesions and squamous carcinomas in old age mice [28]. This supports the idea that K14 expression in airway epithelial cells is a marker of a self-renewing progenitor cell, and is a putative tumor-initiating cell, which requires genetic and/or epigenetic changes in order to be sufficient for carcinogenesis. While we found no difference in the proliferative capacity of K14-expressing cells compared to non-K14-expressing cells in premalignant lesions and in NSCLC, it is possible that the K14+ cells are an important subset of tumor cells as the keratin 14 cytoskeletal protein may allow for changes in cell shape and motility with an increased potential for cell migration. We did, however, find that K14 transiently overexpressing BEAS-2B cells had increased motility compared to control transfected cells in a wound healing assay (FIG. 12).

In summary, the presence of K14+ cells in NSCLC is a biomarker of tumors with a worse prognosis. The presence of K14+ cells is especially predictive in smokers, and furthermore is associated with an increased likelihood of metastases in these patients.

### REFERENCES

1. Jemal A, Siegel R, Ward E, et al. Cancer statistics, 2008. CA Cancer J Clin 2008; 58:71-96.
2. van Klayeren R J, van't Westeinde S C, de Hoop B J, Hoogsteden H C. Stem cells and the natural history of lung cancer: implications for lung cancer screening. Clin Cancer Res 2009; 15:2215-8.
3. McDonald S A, Graham T A, Schier S, Wright N A, Alison M R. Stem cells and solid cancers. Virchows Arch 2009; 455:1-13.
4. Ailles L E, Weissman I L. Cancer stem cells in solid tumors. Curr Opin Biotechnol 2007; 18:460-6.
5. Boman B M, Wicha M S. Cancer stem cells: a step toward the cure. J Clin Oncol 2008; 26:2795-9.
6. Clarke M F, Dick J E, Dirks P B, et al. Cancer stem cells—perspectives on current status and future directions: AACR Workshop on cancer stem cells. Cancer Res 2006; 66:9339-44.
7. Huntly B J, Gilliland D G. Cancer biology: summing up cancer stem cells. Nature 2005; 435:1169-70.
8. Rosen J M, Jordan C T. The increasing complexity of the cancer stem cell paradigm. Science 2009; 324:1670-3.
9. Visvader J E, Lindeman G J. Cancer stem cells in solid tumours: accumulating evidence and unresolved questions. Nat Rev Cancer 2008; 8:755-68.
10. Ward R J, Dirks P B. Cancer stem cells: at the headwaters of tumor development. Annu Rev Pathol 2007; 2:175-89.
11. Kim C F, Dirks P B. Cancer and stem cell biology: how tightly intertwined? Cell Stem Cell 2008; 3:147-50.
12. Rock J R, Onaitis M W, Rawlins E L, et al. Basal cells as stem cells of the mouse trachea and human airway epithelium. Proc Natl Acad Sci USA 2009; 106:12771-5.
13. Schoch K G, Lori A, Burns K A, Eldred T, Olsen J C, Randell S H. A subset of mouse tracheal epithelial basal cells generates large colonies in vitro. Am J Physiol Lung Cell Mol Physiol 2004; 286:L631-42.
14. Hong K U, Reynolds S D, Watkins S, Fuchs E, Stripp B R. In vivo differentiation potential of tracheal basal cells: evidence for multipotent and unipotent subpopulations. Am J Physiol Lung Cell Mol Physiol 2004; 286:L643-9.
15. Engelhardt J F, Schlossberg H, Yankaskas J R, Dudus L. Progenitor cells of the adult human airway involved in submucosal gland development. Development 1995; 121:2031-46.
16. Hong K U, Reynolds S D, Watkins S, Fuchs E, Stripp B R. Basal cells are a multipotent progenitor capable of renewing the bronchial epithelium. Am J Pathol 2004; 164:577-88.
17. Lloyd C, Yu Q C, Cheng J, et al. The basal keratin network of stratified squamous epithelia: defining K15 function in the absence of K14. J Cell Biol 1995; 129:1329-44.
18. Gomperts B N, Kim U, Flaherty S A, Hackett B P. IL-13 regulates cilia loss and foxj1 expression in human airway epithelium. Am J Respir Cell Mol Biol 2007; 37:339-46.
19. Mah V, Seligson D B, Li A, et al. Aromatase expression predicts survival in women with early-stage non small cell lung cancer. Cancer Res 2007; 67:10484-90.
20. Belperio J A, Keane M P, Burdick M D, et al. Role of CXCR2/CXCR2 ligands in vascular remodeling during bronchiolitis obliterans syndrome. J Clin Invest 2005; 115:1150-62.
21. Genden E M, Iskander A, Bromberg J S, Mayer L. The kinetics and pattern of tracheal allograft re-epithelialization. Am J Respir Cell Mol Biol 2003; 28:673-81.
22. Bild A H, Yao G, Chang J T, et al. Oncogenic pathway signatures in human cancers as a guide to targeted therapies. Nature 2006; 439:353-7.
23. Bhattacharjee A, Richards W G, Staunton J, et al. Classification of human lung carcinomas by mRNA expression profiling reveals distinct adenocarcinoma subclasses. Proc Natl Acad Sci USA 2001; 98:13790-5.
24. Cornfield J, Haenszel W, Hammond E C, Lilienfeld A M, Shimkin M B, Wynder E L. Smoking and lung cancer: recent evidence and a discussion of some questions. Int J Epidemiol 2009.
25. Kim C F, Jackson E L, Woolfenden A E, et al. Identification of bronchioalveolar stem cells in normal lung and lung cancer. Cell 2005; 121:823-35.
26. Salnikov A V, Gladkich J, Moldenhauer G, Volm M, Mattern J, Herr I. CD133 is indicative for a resistance phenotype but does not represent a prognostic marker for survival of non-small cell lung cancer patients. Int J Cancer 2009.
27. Hosen N, Park C Y, Tatsumi N, et al. CD96 is a leukemic stem cell-specific marker in human acute myeloid leukemia. Proc Natl Acad Sci USA 2007; 104:11008-13.
28. Dakir E L, Feigenbaum L, Linnoila R I. Constitutive expression of human keratin 14 gene in mouse lung induces premalignant lesions and squamous differentiation. Carcinogenesis 2008; 29:2377-84.
29. Giangreco A, Groot K R, Janes S M. Lung cancer and lung stem cells: strange bedfellows? Am J Respir Crit Care Med 2007; 175:547-53.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of providing a prognosis for a lung cancer in a subject, the method comprising the steps of:
(a) analyzing a sample from the subject with an assay that specifically detects keratin 14 (K14)-expressing cells; and
(b) determining whether the number of K14-expressing cells in the sample is at least 5% of the total number of cells in the sample; and thereby providing the prognosis for the lung cancer.

2. (canceled)

3. The method of claim 1, wherein the lung cancer is a non-small cell lung cancer.

4. The method of claim 1, wherein the lung cancer is a squamous cell carcinoma.

5. The method of claim 1, wherein the assay is flow cytometry, immunofluorescence, or immunohistochemistry.

6. (canceled)

7. The method of claim 1, wherein the assay detects a K14-expressing cell that also expresses keratin 5 (K5).

8. The method of claim 1, wherein the sample is from lung tissue, a lung tumor biopsy, a lymph node biopsy, an adrenal tumor biopsy, a liver tumor biopsy, a brain tumor biopsy, or a bone tumor biopsy.

9. The method of claim 1, wherein the subject has a history of smoking.

10. (canceled)

11. (canceled)

12. (canceled)

13. (canceled)

14. (canceled)

15. (canceled)

16. (canceled)

17. (canceled)

18. (canceled)

19. (canceled)

20. A method of predicting the risk of metastasis of a lung cancer in a subject, the method comprising the steps of:
(a) analyzing a sample from the subject with an assay that specifically detects keratin 14 (K14)-expressing cells; and
(b) determining whether or not the number of K14-expressing cells in the sample is increased as compared to a control; thereby predicting the risk of metastasis of the lung cancer.

21. The method of claim 20, wherein an increased number of K14-expressing cells in the sample as compared to the control indicates a higher risk of metastasis of the lung cancer.

22. The method of claim 20, wherein the lung cancer is a non-small cell lung cancer.

23. The method of claim 22, wherein the cancer is a squamous cell carcinoma.

24. The method of claim 20, wherein the assay detects protein and is ELISA, Western blotting, flow cytometry, immunofluorescence, immunohistochemistry, or mass spectroscopy.

25. The method of claim 20, wherein the assay detects nucleic acid and is mass spectroscopy, PCR, microarray hybridization, thermal cycle sequencing, capillary array sequencing, or solid phase sequencing.

26. The method of claim 20, wherein the sample is from lung tissue, a lung tumor biopsy, a lymph node biopsy, an adrenal tumor biopsy, a liver tumor biopsy, a brain tumor biopsy, or a bone tumor biopsy.

27. The method of claim 20, wherein the subject has a history of smoking.

28. (canceled)

29. (canceled)

30. (canceled)
